(19) United States
(12) Patent Application Publication
(10) Pub. No.: US 2020/0170832 A1
(43) Pub. Date: Jun. 4, 2020

(54) THERMAL CONTRAST THERAPY SYSTEMS, DEVICES, AND METHODS

(71) Applicant: CASCADE WELLNESS TECHNOLOGIES, INC., Sunriver, OR (US)

(72) Inventors: David Schaefer, Sunriver, OR (US); Richard Smith, Sunriver, OR (US)

(21) Appl. No.: 16/207,923

(22) Filed: Dec. 3, 2018

(51) Int. Cl. A61F 7/00; A61H 9/00

(52) U.S. Cl. CPC A61F 7/00; A61H 9/0007; A61F 2007/0225; A61F 2007/0096; A61H 2230/065; A61F 2007/0054

(57) ABSTRACT

The present disclosure relates to thermal contrast therapy systems, and automated thermal contrast therapy devices configured to interact with such systems and to perform customized thermal contrast therapy treatment sequences. Treatments may be prescribed by a treatment provider or selected by a user of a thermal contrast therapy device associated with such a system. In some embodiments, a thermal contrast therapy device may be configured to receive an indication from one or more temperature sensors and/or flow meters and to effect a desired measure of heat transfer during treatment, for example, by automatically adjusting the temperature and/or flow rate of the heat transfer fluid. In some embodiments, a thermal contrast therapy device may be configured to receive an indication of one or more physiological parameter values and to perform a customized thermal contrast therapy treatment sequence based, at least in part, on the one or more physiological parameter values.

### RELATED APPLICATIONS

This application is a continuation of U.S. patent application Ser. No. 15/336,150, filed Oct. 27, 2016, which is a divisional application of U.S. patent application Ser. No. 14/340,904, filed Jul. 25, 2014, which claims priority to U.S. Provisional Application Ser. No. 62/028,952 filed Jul. 25, 2014, the complete disclosures of which are hereby incorporated by reference into this application.

### BACKGROUND

The present disclosure relates to thermal contrast therapy systems, thermal contrast therapy devices, and methods for providing thermal contrast therapy.

Thermal contrast therapy comprises therapeutic treatments in which a sequence of alternating cooling periods and heating periods are provided to one or more areas of a person's body sought to be treated. The alternating cooling periods and heating periods respectively deliver or remove heat from the treatment area. The treatment sequence may be continued for a specified period of time or a specified number of heating periods and/or cooling periods. For example, the sequence may continue for an amount of time effective to achieve a desired therapeutic result.

Without wishing to be constrained by theory, it is believed that thermal contrast therapy results in cycles of vasodilation and vasoconstriction, thus creating a pumping action which improves blood circulation and quality. Applying heat to body tissue may cause vasodilation, or dilation or widening of blood vessels. When blood vessels dilate, the flow of blood is increased due to a decrease in vascular resistance. Therefore, dilation of arterial blood vessels (mainly the arterioles) is believed to decrease blood pressure. Applying cold to body tissue may cause vasoconstriction, or contraction or narrowing of blood vessels. When blood vessels constrict, the flow of blood is restricted or decreased, forcing blood to move on to other parts of the body.

Blood delivers oxygen and nutrients to and removes wastes from tissues and organs. If circulation is poor or slow, delivery of healing nutrients or removal of toxins may be inadequate or suboptimum, which may cause degeneration of the tissues or organs. By improving the circulation and quality of blood, it is believed that more nutrients are available for cells to use and toxins may be managed more efficiently.

Additionally, lymph vessels contract when exposed to cold, and relax in response to heat. The lymph system, unlike the circulatory system, lacks a central pump. Without wishing to be constrained by theory, it is believed that alternating periods of heating and cooling causes lymph vessels, to dilate and contract to essentially “pump” and move fluid from one area to another. This movement of fluid positively affects the inflammation process, which is the body's primary mechanism for healing damaged tissue.

Given these and other physiological effects known in the art, thermal contrast therapy may be an effective treatment for a number of different conditions. The therapy may be applied to any part of the body, for example, a part of the body that is inflamed, congested, injured, or fatigued, or recovering from a surgical procedure. General therapeutic uses of thermal contrast therapy include management of pain, swelling, fever, toxins, spasms, constipation, and immune function. Thermal contrast therapy may be used generally to reduce edema or swelling, reduce or sooth pain, and encourage healing of bones and tissue, and rehabilitate injuries to bone, muscle, ligaments, tendons, and skin. Thermal contrast therapy may also be an effective treatment for patients with hypothermia or frostbite, for example, to enhance circulation in affected tissue.

Thermal contrast therapy may also be used after an acute injury or surgery to reduce pain and swelling, and to promote healing. Athletes have also used thermal contrast therapy after or between training sessions to speed recovery or reduce delayed onset muscle soreness, by helping to flush lactic acid from sore muscles. Thermal contrast therapy may also be used to relax joint tissue, such as ligaments and tendons, to increase range of motion. Thermal contrast therapy may also be an effective treatment for patients with spinal cord or nerve damage, for example, to enhance blood flow to the injured nerve tissue, or prevent or mitigate disputation of the tenuous blood supply.

Treatments may also be an effective therapy for diabetes and related physiological complications, for example, gangrene, or for lymphedema or other disorders associated with vascular or lymphatic insufficiency, for example, Chronic Venous Insufficiency, venous stasis ulcers, post-mastectomy edema or chronic lymphedema, and for peripheral vascular disease or any other circulatory deficiency syndrome, for example, arteriosclerosis, deep vein thrombosis, Buereger's disease, or thromboangiitis obliterans. Other studies indicate that thermal contrast therapy may positively influence the immune system. Treatments may also be an effective therapy for destruction of subcutaneous lipid cells.

Thermal contrast therapy may be performed manually, such as by alternately immersing a target area of the body (e.g., a foot, ankle, or leg) in a cold water whirlpool or bath and a warm whirlpool, or by alternately applying a heat source, such as a heating pad or hydrocollator pack, and a cold source, such as an ice pack, or the like.

Alternatively, thermal contrast therapy devices are commercially available which circulate water or other fluid through a treatment cuff surrounding the target area of the body. These devices may be cumbersome to operate and may be unsuitable for personal use, such as in-home treatments. It is believed that the effectiveness of thermal contrast therapy is dependent on the ease with which the therapy may be applied. If self-treatment is cumbersome or uncomfortable, a patient may be less likely to undergo treatment, and the opportunity to attain the desired therapeutic result may be diminished. It would be advantageous to provide improved thermal contrast therapy systems, devices, and methods as described herein, including features that simplify operation, provide for more user-friendly treatments, and generally improve user experience.

Existing thermal contrast therapy devices typically provide pre-programmed therapies, with only limited ability to vary parameters of the treatment sequence. Because of these and other limitations, existing thermal contrast therapy devices may frequently provide a suboptimum treatment, for example, when the pre-programmed sequence differs from the ideal treatment. In some situations, a treatment provider or a user may administer treatments which may actually be harmful to the body. For example, if too much heating or cooling is provided, blood flow may be detrimentally restricted and/or tissue may be damaged. Such blood flow restriction may arise from blood pooling caused by excessive heating, or from constriction of blood vessels caused by excessive cooling.

It is believed that the effectiveness of thermal contrast therapy is dependent on the level of accuracy, precision, and control of various parameters of the treatment sequence. For example, the effectiveness may depend on the ability to administer and control specifically prescribed fluid temperatures, flow rates, rates of heat transfer, number of heating periods and cooling periods an durations thereof, total therapy duration, and other variables. Available devices may not provide the ideal treatment. The effectiveness of thermal contrast therapy may be enhanced by providing systems, methods, and devices in accordance with the present disclosure. Treatment sequences may be optimized, for example, based on characteristics of a particular patient, physiological parameter values exhibited by a patient, a particular area of the body being treated, a particular symptom or condition sought to be treated, a particular desired therapeutic result, or other factors. Thus, it would be advantageous to provide improved thermal contrast therapy systems, devices, and methods as described herein, including features that provide greater variability of treatment parameters, enhanced treatment precision and accuracy, and the ability to provide customized treatments.

The skilled artisan will appreciate that research into the effects and benefits of thermal contrast therapy under various treatment conditions is relatively underdeveloped, and in many instances practitioners may desire further research as to the best way to treat a particular patient, body part, injury, ailment, or condition. It would be advantageous to provide researchers and practitioners with systems, devices, and methods that facilitate further research into alternative thermal contrast therapy treatment sequences.

Thermal contrast therapy may be provided to any human or other mammalian patient, and as such, the present disclosure has applicability in the medical and veterinary arts. The artisan will appreciate that references in the present disclosure to a person, patient, user, and/or recipient of thermal contrast therapy treatments may also means and refer to any other mammalian species.

### SUMMARY

A summary of certain embodiments disclosed herein is set forth below. It should be understood that these aspects are presented merely to provide the reader with a brief summary of these certain embodiments and that they are not intended to limit the scope of the present disclosure.

The present disclosure relates to thermal contrast therapy systems configured to provide customized thermal contrast therapy, automated thermal contrast therapy devices, and computer-implemented methods of providing thermal contrast therapy. It should be understood that the thermal contrast therapy systems, devices, and methods disclosed herein may be implemented alone, together, or in combination with one another or other systems, devices, or methods known in the art.

The thermal contrast therapy systems disclosed herein include systems comprising a thermal contrast therapy network, and one or more thermal contrast therapy devices configured to transmit data to and receive data from the thermal contrast therapy network. The thermal contrast therapy devices may be associated with one or more users, and the system may be configured to provide customized thermal contrast therapy to the users. In some embodiments, customized thermal contrast therapy treatment programs may be automatically generated based on data housed in a database associated with a thermal contrast system.

The thermal contrast therapy devices disclosed herein include devices comprising a feed pump configured to circulate fluid through a treatment cuff to provide thermal contrast therapy treatments. In some embodiments, a thermal contrast therapy device may be configured to function with a plurality of different treatment cuffs. For example, a device may be configured to receive an indication effective to identify a treatment cuff operably connected to the device and to cause the device to perform a thermal contrast therapy treatment sequence that is calibrated for the particular treatment cuff operably connected to device.

In some embodiments, a thermal contrast therapy device may be configured to perform a treatment sequence derived, at least in part, from an input received from a thermal contrast therapy system. The treatment sequence may be prescribed by a treatment provider or selected by a user of the thermal contrast therapy device. In accordance with the present disclosure, a treatment sequence may be based on, for example, previously provided treatment sequences, physiological parameter values exhibited by a patient during a previous treatment, and/or therapeutic effects observed in a patient during a previous treatment.

The thermal contrast therapy devices disclosed herein may be configured to automatically adjust one or more sequence parameters so as to perform a selected treatment sequence. In some embodiments, a thermal contrast therapy device may be configured to allow a user to select or input customized sequence parameters, which may include, among other things, a customized time duration, pressure pulse frequency of the heat transfer fluid, cuff compression, temperature-change profile, fluid temperature, and/or flow rate corresponding to one or more of the periods. In some embodiments, a thermal contrast therapy device may be configured to receive an indication from one or more temperature sensors and/or flow meters and to effect a desired measure of heat transfer between the fluid and the patient, for example, by automatically adjusting the temperature and/or flow rate of the heat transfer fluid. In some embodiments, a thermal contrast therapy device may be configured to receive an indication of one or more physiological parameter values exhibited by a patient and to perform a customized thermal contrast therapy treatment sequence based, at least in part, on the one or more physiological parameter values.

In some embodiments, a thermal contrast therapy device may be configured to transmit data to or receive data from a thermal contrast therapy system. Such data may include, for example, physiological parameter values exhibited by a patient, for example, during treatment. In some embodiments, subsequent additional treatments may be provided based, at least in part, on data corresponding to previous treatments.

The methods disclosed herein include computer-implemented methods of providing thermal contrast therapy. In some embodiments, computer-implemented methods of providing thermal contrast therapy include circulating fluid from a thermal contrast therapy device through a treatment cuff, the treatment cuff having been applied to a patient, while providing a sequence comprising a plurality of alternating cooling periods and heating periods. Any one or more variables in the thermal contrast therapy sequence may be modified in order to provide a customized thermal contrast therapy treatment sequence in accordance with the present disclosure. As examples, a customized treatment sequence may include automatically changing the fluid temperature and/or flow rate, the duration of one or more periods, the number of periods, and/or prescribing a sequence for one or more future thermal contrast therapy treatments.

A customized thermal contrast therapy treatment sequence may correspond to a treatment program having been prescribed by a treatment provider or selected by a user of a thermal contrast therapy device. The program may be selected, for example, from a database associated with a thermal contrast therapy system configured to provide customized thermal contrast therapy treatment programs. A thermal contrast therapy treatment program may comprise customized sequence parameters based, for example, on a physiological parameter value exhibited by the patient, an area of the body sought to be treated, a particular symptom or condition sought to be treated, and/or a particular desired therapeutic result.

In some embodiments, a customized thermal contrast therapy treatment sequence includes effecting a desired measure of heat transfer between the fluid and the patient during one or more of the cooling periods and heating periods. The desired measure of heat transfer may be effected by automatically adjusting the flow rate of the fluid and/or the temperature of the fluid.

In some embodiments, a thermal contrast therapy treatment sequence may include rapidly alternating cooling periods and heating periods. Such a sequence may be effective to induce cycles of vasoconstriction and vasodilation. Cycles of vasoconstriction and vasodilation may be provided in a manner effective to cause an increase in blood circulation and/or blood oxygen content in a patient's tissue proximate to the treatment cuff during thermal contrast therapy.

In some embodiments, a thermal contrast therapy treatment sequence may be automatically adjusted to optimize one or more physiological parameters. For example, a thermal contrast therapy treatment sequences may be automatically adjusted so as to optimize the magnitude of an increase in blood circulation and/or blood oxygen content induced by cycles of vasoconstriction and vasodilation. In some embodiments, a thermal contrast therapy treatment sequence may be automatically adjusted when the patient exhibits a physiological parameter value that corresponds to a predefined value

In some embodiments, automatic adjustments to a thermal contrast therapy treatment sequence may include: changing a setting for the fluid temperature and/or flow rate; changing the duration of one or more of the cooling periods, heating periods, and/or transition periods; changing the number of cooling periods, heating periods, and/or transition periods; changing the frequency of the pressure pulse of the fluid; changing the compression of the treatment cuff; changing the rate of change of the temperature and/or flow rate of the fluid; and/or prescribing a sequence for one or more future thermal contrast therapy treatments.

In accordance with the present disclosure, one or more computer-readable media bearing computer-readable instructions may be provided, that, when executed by a processor such as in an automated thermal contrast therapy device, cause the device to perform one or more of the computer-implemented methods of providing thermal contrast therapy which are disclosed herein. In addition to the foregoing, various other systems, devices, and methods, and non-transitory computer-readable media are set forth and described in the teachings of the present disclosure.

The foregoing summary may contain simplifications, generalizations, inclusions, and/or omissions of detail; consequently, those skilled in the art will appreciate that the summary is illustrative only and is not intended to be in any way limiting. In addition to the illustrative aspects, embodiments, and features described above, further aspects, embodiments, and features will become apparent by reference to the drawings, the following detailed description of certain embodiments, and the claims.

### DESCRIPTION OF THE DRAWINGS

FIG. 1 shows an exemplary schematic of a thermal contrast therapy system.

FIG. 2A shows a schematic of an exemplary thermal contrast therapy device.

FIG. 2B shows a schematic of an exemplary thermal contrast therapy device, with the addition of a neutral reservoir and related aspects.

FIG. 3A shows a flow chart illustrative of an exemplary thermal contrast therapy treatment process for providing a desired measure of heat transfer.

FIG. 3B shows a flow chart illustrative of an exemplary thermal contrast therapy treatment process for providing a desired measure of heat transfer, utilizing flow rate to control the rate of heat transfer.

FIG. 4 shows a flow chart illustrative of an exemplary customized thermal contrast therapy treatment process, dynamically controlled to optimize a physiological parameter value.

FIG. 5A shows an exemplary customized thermal contrast therapy treatment sequence configured to provide a desired fluid temperature during the various periods, for a first treatment of an athletic injury to a knee or elbow in a healthy/strong patient within the first 24 hours after the injury, to reduce inflammation.

FIG. 5B shows an exemplary customized thermal contrast therapy treatment sequence configured to provide a desired fluid temperature during the various periods, for subsequent treatment of an athletic injury to a knee or elbow in a healthy/strong patient after the first 24 hours following the injury, to reduce inflammation and increase blood flow.

FIG. 5C shows an exemplary customized thermal contrast therapy treatment sequence configured to provide a desired fluid temperature during the various periods, for ongoing treatment of an athletic injury to a knee or elbow in a healthy/strong patient after the first 48 hours following the injury, to reduce inflammation, increase blood flow, and for pain management.

FIG. 5D shows an exemplary customized thermal contrast therapy treatment sequence configured to provide a desired fluid temperature during the various periods, for ongoing treatment of gangrene below the knee in an elderly patient, to increase blood flow and revitalize tissue.

FIG. 5E shows an exemplary customized thermal contrast therapy treatment sequence configured to provide a desired measure of heat transfer during the various periods, for a first treatment of an athletic injury to a knee or elbow in a healthy/strong patient within the first 24 hours after the injury, to reduce inflammation.

FIG. 5F shows an exemplary customized thermal contrast therapy treatment sequence configured to provide a desired measure of heat transfer during the various periods, for subsequent treatment of an athletic injury to a knee or elbow in a healthy/strong patient after the first 24 hours following the injury, to reduce inflammation and increase blood flow.

FIG. 5G shows an exemplary customized thermal contrast therapy treatment sequence configured to provide a desired measure of heat transfer during the various periods, for ongoing treatment of an athletic injury to a knee or elbow in a healthy/strong patient after the first 48 hours following the injury, to reduce inflammation, increase blood flow, and for pain management.

FIG. 6 shows an exemplary user or patient profile page for a thermal contrast therapy device or system.

FIG. 7A shows an exemplary user interface for a treatment provider to utilize a thermal contrast therapy system.

FIG. 7B shows an exemplary user interface for a treatment provider to manage patients receiving thermal contrast therapy treatments via a thermal contrast therapy system.

FIG. 7C shows an exemplary user interface for a treatment provider to manage thermal contrast therapy treatments prescribed to a patient via a thermal contrast therapy system.

FIG. 7D shows an exemplary user interface for searching a thermal contrast therapy system database for customized thermal contrast therapy treatments.

FIG. 7E shows an exemplary user interface for operation of thermal contrast therapy device providing a selection of manual or preprogrammed operation.

FIG. 7F shows an exemplary user interface for a user to cause a thermal contrast therapy device to perform a customized thermal contrast therapy treatment.

FIG. 7G shows an exemplary user interface for manual operation of thermal contrast therapy device.

### DETAILED DESCRIPTION OF ILLUSTRATIVE EMBODIMENTS

In the following detailed description of illustrative embodiments, thermal contrast therapy systems, thermal contrast therapy devices, and methods for providing thermal contrast therapy will be described in greater detail with reference to several embodiments thereof as disclosed herein and illustrated in the accompanying figures. In the following detailed description of illustrative embodiments, numerous specific details are set forth in order to provide a thorough understanding of the disclosed systems, devices, and methods. It will be apparent, however, to one skilled in the art, that the presently disclosed systems, devices, and methods may be practiced without some or all of these specific details. In other instances, well known process steps and/or structures have not been described in detail in order to not unnecessarily obscure the present disclosure.

The following detailed description of illustrative embodiments is therefore not to be taken in a limiting sense, and it is intended that other embodiments are within the scope of the presently disclosed devices, method, and systems. The features and advantages of the presently disclosed systems, devices, and methods may be better understood with reference to the figures and discussions that follow. The claimed subject matter is defined by the appended claims and their equivalents.

### I. Thermal Contrast Therapy Systems

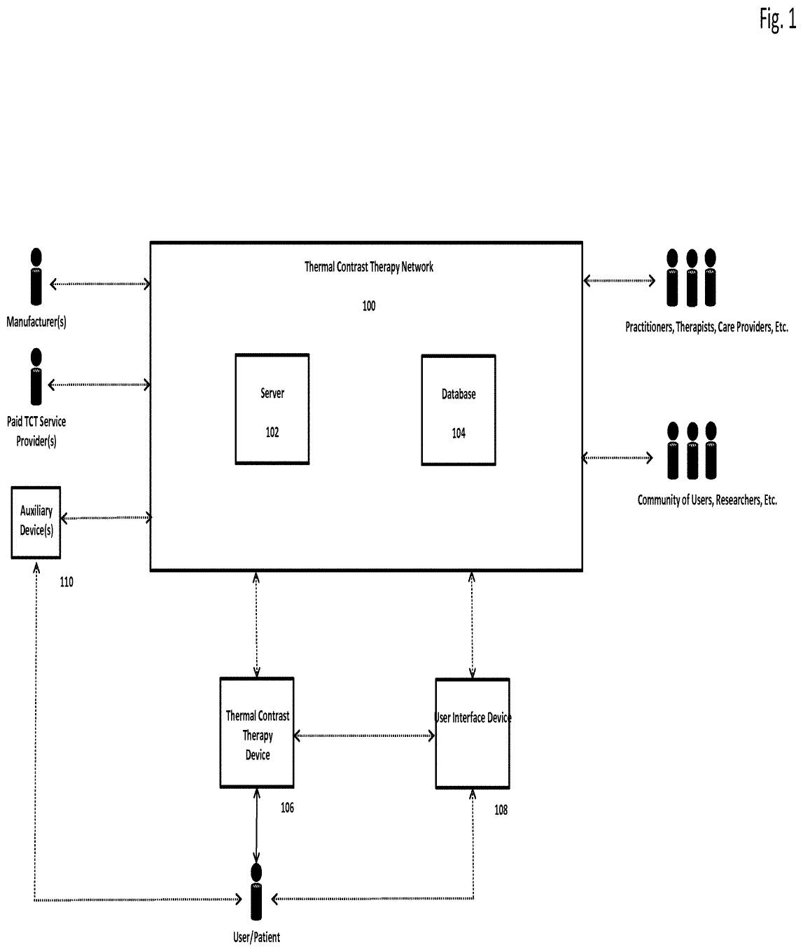

The present disclosure relates to, among other things, thermal contrast therapy systems configured to provide customized thermal contrast therapy, for example via automated thermal contrast therapy devices configured to interact with such systems and to perform computer-implemented methods of providing thermal contrast therapy. Referring to FIG. 1, an exemplary embodiment of a thermal contrast therapy system is shown. Those skilled in the art will appreciate that numerous other embodiments of thermal contrast therapy systems are within the spirit and scope of the present disclosure.

The thermal contrast therapy system shown in FIG. 1 depicts a thermal contrast therapy network 100 comprising a server 102, and a database 104. The server may be configured to manage network connections, cause data to be stored and/or retrieved from the database, and transmit and receive data through various sources via network connections. One or more thermal contrast therapy devices 106 may be associated with a thermal contrast therapy system. These thermal contrast therapy devices may transmit data to and receive data from the thermal contrast therapy network. Connectivity may be accomplished through means well known in the art, including wired or wireless connections, including USB type connections, Bluetooth, and Wi-Fi.

Data may be stored in the database and subsequently transmitted to a thermal contrast therapy device. Such data may include treatment programs, for example customized treatment programs such as those disclosed herein. In some embodiments, a thermal contrast therapy treatment program may be uploaded or stored in a database accessible by a thermal contrast therapy system or device, and users thereof. The treatment program may be prescribed by a user's therapist, physician, care provider, or the like, or selected by a user, for example, from a menu of one or more treatment options.

Treatment programs may be developed by thermal contrast therapy treatment providers, for example therapists, physicians, care providers, and the like, researchers, manufacturers, or service providers, for example providers of paid or subscription-based thermal contrast therapy services, users of a thermal contrast therapy device or system, or any other party associated with the thermal contrast therapy system. As described in more detail below, thermal contrast therapy treatment programs may be associated with various patient profiles, particular areas of the body sought to be treated, particular symptom or conditions sought to be treated, particular desired therapeutic results, and/or other considerations.

Data may be transmitted across a thermal contrast therapy network and stored in the database. For example, data associated with a thermal contrast therapy treatment may be transmitted from a thermal contrast therapy device, a personal device configured to operate a thermal contrast therapy device (e.g., via a user interface), an auxiliary device configured to monitor physiological parameter values, and/or any other device. Databases associated with the thermal contrast therapy systems disclosed herein may house data such as: information related to a patient receiving treatment, thermal contrast therapy treatment programs and treatment sequences, physiological parameter values exhibited by patients (e.g., during treatments), and therapeutic effects observed in patients (e.g., following treatments), or other desired information. In some embodiments, customized thermal contrast therapy treatment programs may be prescribed to the users based, at least in part, on data housed in a database associated with a thermal contrast system. In some embodiments, customized thermal contrast therapy treatment programs may be automatically generated based on such data.

In some embodiments, users may interact with a thermal contrast therapy system or a thermal contrast therapy device via a user interface, e.g., on a user interface device 108. Such use interface may be configured to control a thermal contrast therapy device. For example, a user interface maybe configured to allow a user (e.g., a treatment provider and/or a patient) to select from among a plurality customized thermal contrast therapy treatment programs and to cause a thermal contrast therapy device to perform the selected treatment program. Exemplary embodiments of user interfaces are described in more detail below. In some embodiments, the thermal contrast therapy systems disclosed herein may be configured to allow a user to define a customized thermal contrast therapy treatment sequence and cause the sequence to be stored in a database associated with the system, thereby making the sequence available to one or more of the users of the system. Users of a thermal contrast therapy system in accordance with the present disclosure include patients or other users of thermal contrast therapy devices, treatment providers (e.g., therapists, physicians, care providers, and the like), and communities or groups of researchers, treatment providers, and/or users of thermal contrast therapy devices.

In some embodiments, the thermal contrast therapy systems disclosed herein may include one or more auxiliary devices 110. Such auxiliary devices may be associated with a thermal contrast therapy device and/or thermal contrast therapy network. The auxiliary devices may be configured to measure one or more physiological parameters (e.g., vital signs), and to transmit data corresponding to such physiological parameter values to a thermal contrast therapy device and/or to a thermal contrast therapy network. Physiological parameter values which may be measured by such an auxiliary device include: body temperature (e.g. core temperature, local temperature, etc.), heart rate, blood pressure, blood flow, blood oxygen level, or other vital signs. Such physiological parameter values may be monitored using any number of devices which are well known in the art. In some embodiments, a thermal contrast therapy device may be configured to provide customized thermal contrast therapy treatment sequences based, at least in part, on data from an auxiliary device.

In some embodiments, the thermal contrast therapy systems disclosed herein may be configured to provide thermal contrast therapy prescribed to a patient from a remote location, and/or to remotely manage thermal contrast therapy to be performed at any one or more of a plurality of locations. As examples, such locations may include: a hospital, an urgent care facility, a medical clinic, a physical therapy clinic, a chiropractic clinic, a naturopathic clinic, an acupuncture facility, a sports facility, a gym, and a patient's home.

In some embodiments, a treatment provider may prescribe a thermal contrast therapy program for a patient, upload the program to a database associated with a thermal contrast therapy system where it will be accessible by the patient and/or the patient's thermal contrast therapy device, for example, via a user interface. The patient may then administer the prescribed thermal contrast therapy treatment, for example at home or any other location. When a patient administers a thermal contrast therapy treatment, data associated with treatment may be stored in the database where it may be accessible by the patient's treatment provider, who may review the data and provide ongoing treatments. Subsequent treatments may be modified based on the results of previous treatment and/or the treatment provider's review of such data. Similarly, paid or subscription-based service providers may review treatment data and provide ongoing subscription-based treatments or other services.

In some embodiments, a thermal contrast therapy system may be configured to administer safety protocols, for example to ensure that treatments are properly administered. Thermal contrast therapy devices may require access credentials in order for the device to function. In some embodiments, access credentials may define or limit the functionality of the device, for example the range of operation, availability of features, treatment programs which may be administered, frequency of treatments, etc. These access credentials may be assigned to a user by a treatment provider, manufacturer or service provider, or established by a user when creating a user profile.

In some embodiments, a thermal contrast therapy system may be accessible by manufacturers, service providers, and/or other third parties. These parties may provide customer service and technical support, diagnostics, updates and upgrades, off-site programming and monitoring of devices, paid or subscription/lease-based services (e.g., for device rentals and/or treatment services), selections of thermal contrast therapy treatment programs accessible by users and/or treatment providers, and various other services. Where thermal contrast therapy is provided to users on a subscription basis, the thermal contrast therapy systems disclosed herein may be configured to control the functionality of a user's thermal contrast therapy device based, at least in part, on the status of the user's subscription.

### II. Thermal Contrast Therapy Devices

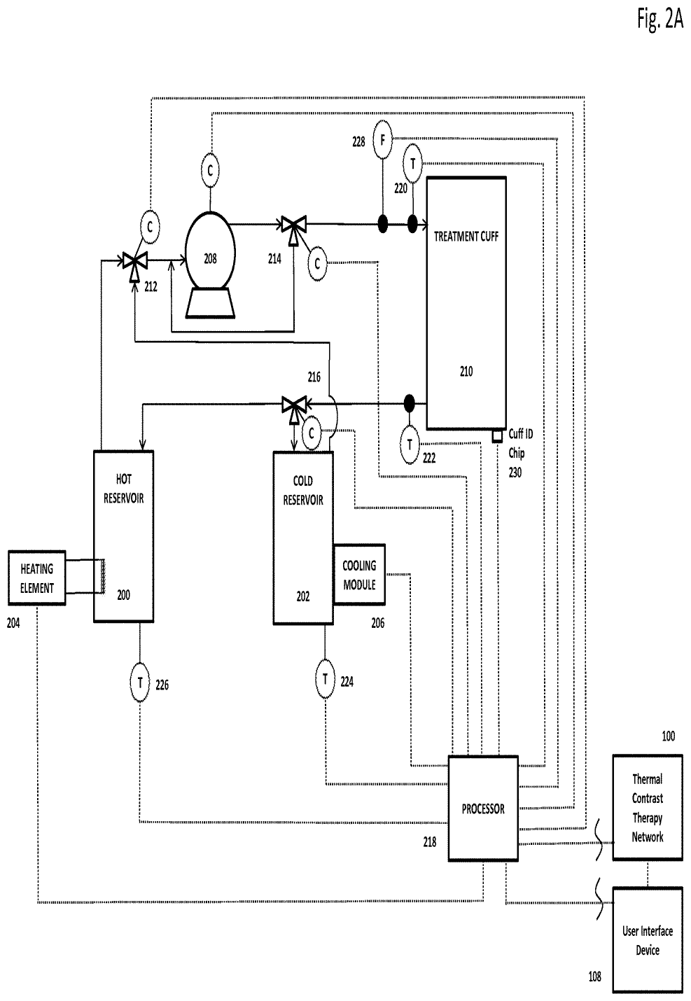

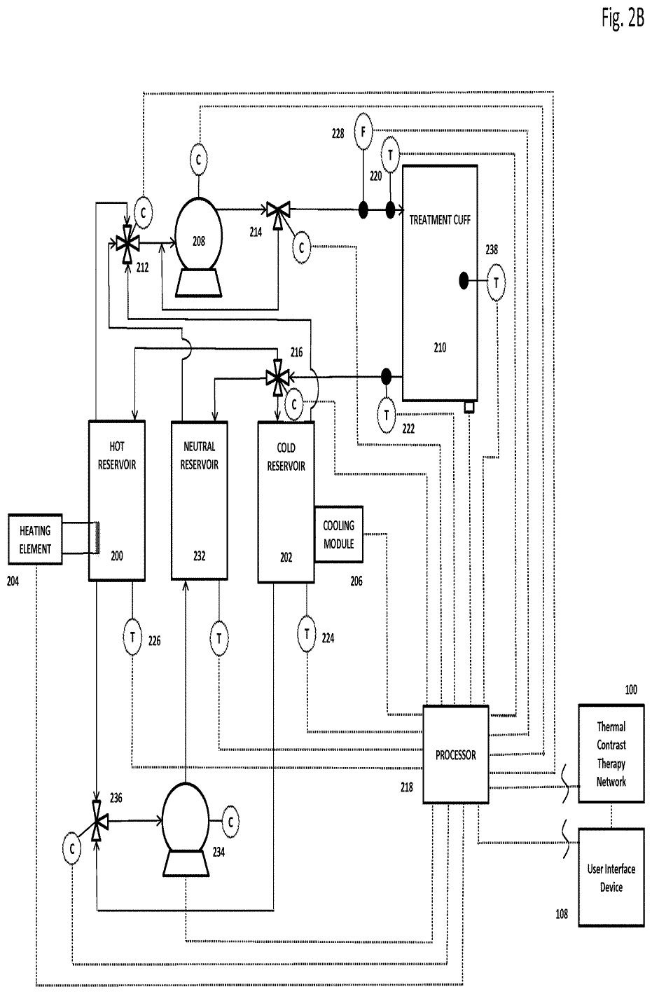

Referring to FIG. 2A, a schematic of an exemplary thermal contrast therapy device is shown. Those skilled in the art will appreciate that numerous other embodiments of thermal contrast therapy devices are within the spirit and scope of the present disclosure.

The thermal contrast therapy device shown in FIG. 2A comprises a hot reservoir 200 configured to store heat transfer fluid for heating periods, a cold reservoir 202 configured to store heat transfer fluid for cooling periods, a heating element 204 configured to heat the fluid in the hot reservoir, and a cooling module 206 configured to cool the fluid in the cold reservoir. A feed pump 208 circulates heat transfer fluid from the respective fluid reservoirs to one or more treatment cuffs 210. The heat transfer fluid flows through the treatment cuff and back to one of the fluid reservoirs.

Fluid is directed to the feed pump by one or more feed control valves 212. The flow rate of the fluid delivered to the one or more treatment cuffs is regulated by one or more flow control valves/flow regulators 214. Fluid exiting the treatment cuff is directed to a designed fluid reservoir by one or more return control valves 216.

As further depicted in FIG. 2A, thermal contrast therapy devices may be equipped with a processor 218 to control the operation of device. The processor may be configured to cause the thermal contrast therapy device to perform customized thermal contrast therapy treatment sequences, by monitoring and/or controlling various aspects of the device, including the heating element, cooling module, pump(s), and valve(s).

In some embodiments, a thermal contrast therapy device may be equipped with one or more temperature sensors and/or flow meters. For example, as depicted in FIG. 2A, temperature sensors 220 and 222 are positioned at the inflow and/or outflow lines to and from the treatment cuff, respectively, and temperature sensors 224 and 226 are positioned at the cold reservoir and the hot reservoir, respectively. As additionally depicted in FIG. 2A, flow meter 228 is positioned at the inflow line to the treatment cuff. Processor 218 may be configured to control the temperature of the heat transfer fluid, utilizing temperature measurements from any one or more temperature sensors, and/or the flow rate of the heat transfer fluid, utilizing flow measurements from the flow meter. Such temperature and/or flow control may be configured to enable the device to provide customized thermal contrast therapy treatment sequences based, at least in part, on fluid temperature and/or flow rate in accordance with the present disclosure.

In some embodiments, the processor may be configured to interact with a thermal contrast therapy network 100 as described in the present disclosure. For example, the processor may be configured to perform a treatment sequence derived, at least in part, from an input received from a thermal contrast therapy system. The treatment sequence may be prescribed buy a treatment provider or selected by a user of the thermal contrast therapy device. The selected treatment may be chosen from a database of available treatment sequences on a thermal contrast therapy network, or manually input, via a user interface. The processor may be configured to automatically adjust one or more sequence parameters so as to perform a selected treatment sequence.

In some embodiments, a thermal contrast therapy device may be configured to allow a user to select or input customized sequence parameters, which may include, among other things, a customized time duration, pressure pulse frequency of the heat transfer fluid, cuff compression, temperature-change profile, fluid temperature, and/or flow rate corresponding to one or more of the periods. In some embodiments, the processor 218 may be configured to receive an indication from one or more temperature sensors and/or flow meters and to effect a desired measure of heat transfer between the fluid and the patient, for example, by automatically adjusting the temperature and/or flow rate of the heat transfer fluid. In some embodiments, the processor 218 may be configured to receive an indication of one or more physiological parameter values and to cause the thermal contrast therapy device to perform a customized thermal contrast therapy treatment sequence based, at least in part, on the one or more physiological parameter values.

Power to operate the thermal contrast therapy device may be provided via a low voltage power source, for example, a 12 volt DC power source. Such low voltage power source may be safely converted from AC power using a converter.

### Treatment Cuffs

Thermal contrast therapy is generally administered via one or more treatment cuffs. The thermal contrast therapy devices disclosed herein may be configured to function with a plurality of different treatment cuffs. A treatment cuff generally comprises a bladder configured to hold heat transfer fluid, a pouch configured to receive the bladder, which may be constructed of fabric or other material comfortable to a patient's skin, and an attachment mechanism configured to secure the cuff to a patient's body. The attachment mechanism may be configured to provide adjustable dimensions for the treatment cuff, such as provided by Velcro attachments. Treatment cuff bladders may be constructed of a network of interconnected cells or capillaries, with an inflow port configured to receive heat transfer fluid via an inflow tube, and outflow port configured to return heat transfer fluid via an outflow tube. Various shapes and sizes of treatment cuffs may be provided in order to accommodate different patient sizes and/or different areas of the body, and/or in order to accommodate or integrate with various orthopedic braces, casts, and other devices.

In some embodiments, heat transfer fluid temperature and/or flow rate may be measured at point where fluid enters and/or exits the treatment cuff, for example, at the inflow port and/or the outflow port of the bladder. These temperature and/or flow rate measurements may be utilized in thermodynamic calculations to provide customized thermal contrast therapy treatment sequences in accordance with the present disclosure. Such treatment sequences may include effecting a particular measure of heat transfer (i.e., a particular quantity of heat and/or a particular rate of heat transfer). Temperature may also be measured at the fluid reservoirs, or along any point of the inflow tube and/or outflow tube.

In some embodiments, treatment cuffs may be configured with one or more sensors operably connected with the cuff. For example, temperature sensors 220 and 222, and/or flow meter 228, each as depicted in FIG. 2A, may be operably connected with a treatment cuff. Such treatment cuff sensors may be configured to measure treatment conditions and transmit those measurements to a processor 218 in a thermal contrast therapy device and/or to a thermal contrast therapy system.

The skilled artisan will appreciate that for purposes of measuring the temperature change of the heat transfer fluid between the entrance and exit of the treatment cuff, temperature measurements obtained at or near the inflow port and/or the outflow port of the treatment cuff bladder may be more accurate than measurements obtained at more distant locations. This may be of particular concern to the artisan when performing a treatment sequences requiring a high degree of precision, for example, a treatment sequence which effects a desired measure of heat transfer.

In some embodiments, a thermal contrast therapy device may be configured to receive an indication effective to identify a treatment cuff operably connected to the thermal contrast therapy device and to cause the thermal contrast therapy device to perform a treatment sequence that is calibrated for the particular treatment cuff. By identifying the particular treatment cuff operably connected to the device, a substantially similar treatment sequence may be provided using any one of a plurality of different treatment cuffs, notwithstanding differences in the dimensional or physical characteristics of the cuffs. This includes, for example, effecting a desired measure of heat transfer between the fluid and the patient, using any one of a plurality of different treatment cuffs. As depicted in FIG. 2A, a treatment cuff may be electronically tagged or encoded with an identification number via a cuff ID chip 230. The cuff ID chip may be a microchip, RFID chip, or other suitable component capable of enabling a processor in a thermal contrast therapy device to identify which treatment cuff is attached to the device. Alternatively, a cuff identification number may be input manually, for example via a user interface associated with a thermal contrast therapy device or system.

Once a particular treatment cuff has been identified by a thermal contrast therapy device, the device may adjust its operating parameters to correspond to the characteristics of the particular treatment cuff operably connected to the device. In some embodiments, treatment cuffs having different dimensional or physical characteristics may require different fluid flow rates and/or fluid temperatures in order to deliver desired treatment conditions. For example, some treatment sequences in accordance with the present disclosure may require a different flow rates and/or temperature of the heat transfer fluid in order to effect a desired measure of heat transfer (i.e., a particular quantity of heat and/or a particular rate of heat transfer). Identification of the particular treatment cuff enables the thermal contrast therapy device to provide enhanced accuracy, precision, and control of the treatment sequence, for example, by varying the temperature and/or flow rate of the heat transfer fluid based, at least in part, on characteristics of the treatment cuff having been identified as operably connected to the device. Thus, customized thermal contrast therapy treatment sequences may be provided with consistency across a plurality of different treatment cuffs.

Additionally, in some embodiments, a thermal contrast therapy device may be configured to perform a specified treatment sequence or program based on the particular treatment cuff operably connected to the device. For example, if the device identifies a treatment cuff associated with knee treatments, the device may perform a treatment sequence or program designed to treat knees; or, if the device identifies a cuff associated with the lumbar region, the device may perform a treatment sequence designed to treat the lumbar region. Such specified treatment sequences may be provided based on virtually any treatment cuff associated with virtually any area of the body or condition sought to be treated.

In some embodiments, a treatment cuff may be configured to provide compression during the thermal contrast therapy. Compression enhances the contact between the treatment cuff and the patient's tissue, thereby providing a more efficient heat transfer between the heat transfer fluid and the patient's tissue proximate to the treatment cuff. Such compression may be provided, for example, during at least part of any one of the treatment sequences disclosed herein. For example, compression may be provided during heating periods but not cooling periods, during cooling periods but not heating periods, only during transition periods, during both heating periods and cooling periods, during only a subset of periods, or during all of the periods.

In some embodiments, the level of compression may be varied. For example, the rate of heat transfer between the heat transfer fluid and the patient's tissue proximate to the treatment cuff may be varied by varying the level of compression effected by the treatment cuff. The compression level may be automatically adjusted to effect a desired rate of heat transfer. In some embodiments, pulse compression may be provided by alternating between a plurality of chosen pressure levels, for example, to pulse massage the tissue.

Compression may be provided, for example, via a gas pumped into the treatment cuff from an air hose. Here, the treatment cuff may be configured with an air bladder configured to receive and hold air at a pressure effective to provide the desired level of compression. Air may be provided via any suitable air compressor known in the art.

### Peristaltic Pumps

A thermal contrast therapy device may utilize any one or more pumps known in the art suitable to transfer fluid from the reservoirs to one or more treatment cuffs. In some embodiments, one or more low pressure peristaltic pumps or “tube pumps” may be used. A peristaltic pump is a positive displacement pump comprising a rotor with a plurality of roller (or, shoes, wipers, or lobes) attached to the external circumference of the rotor. The rollers compresses flexible, non-reinforced, extruded tubing fitted around the rotor to pump fluid through the tubing. As the rotor turns, part of the tubing under compression is pinched, thus forcing the fluid to be pumped through the tube. Additionally, as the tube opens to its natural state after the rollers pass, fluid flow is induced into the pump. Peristaltic pumps are well known in the art, and any suitable peristaltic pump may be used. For example, suitable peristaltic pumps include the MITYFLEX 908, Model No. 908-058, commercially available from Anko Products Inc., or the Watson Marlow Bredel Series, Model 313D, coupled with a Crouzet 12 VDC gear motor, commercially available from Watson Marlow.

In some embodiments, customized thermal contrast therapy treatment sequences may be provided which are based, at least in part, on the flow rate of the feed pump 208. The flow rate of a peristaltic pump is determined by several factors, including the tubing inner diameter (higher flow rate with larger inner diameter), the pump head outer diameter (higher flow rate with larger outer diameter), and the rate of rotation of the pump head (higher flow rate with higher rate of rotation).

In some embodiments, a thermal contrast therapy device may be configured to provide a pulsation in the fluid. Such pulsation may be provided during at least part of any one of the treatment sequences disclosed herein. As an example, fluid pumped via a peristaltic pump exhibits a pressure pulse caused by the occlusion and return of the tubing as the rollers pass over the tubing. The frequency of the pressure pulse correlates to the rotational rate of the rollers. Control of the feed pump may be configured to adjust the frequency of the pulsation. Additionally, increasing the number of rollers increases the frequency of the pressure pulse, whereas reducing the number of rollers increases the amplitude of the pressure pulse. In some embodiments, a recirculation line may be utilized in conjunction with a peristaltic pump to control flow independently from the parameters of the peristaltic pump, for example, via one or more flow control valves/flow regulators 214. The flow control valves may also be accompanied by a recirculation line to recirculate a portion of the fluid back to the feed pumps in conjunction with the flow control.

Without wishing to be bound by theory, it is believed that a therapeutic effect may be achieved by synchronizing the frequency of a pressure pulse, for example generated by a peristaltic pump, with patient's heart rate. Accordingly, in some embodiments, a thermal contrast therapy device may be configured to receive an indication of a patient's heart rate, and to provide a pressure pulse having a frequency which is at least approximately synchronized with the patient's heart rate. Such synchronized pressure pulse may be provided during at least part of the treatment sequence. Synchronization may be achieved by measuring the patient's heart rate, for example, with a heart rate monitor auxiliary device, and adjusting the rotational rate of the peristaltic pump rollers.

Without wishing to be bound by theory, it is believed that a therapeutic effect may also be achieved by providing a pressure pulse frequency selected to correspond to a desired heart rate. For example, it is believed that under certain conditions a patient's heart rate may be manipulated by providing a certain pressure pulse frequency to one or more treatment cuffs. A pressure pulse frequency may effect a change in the patient's heart rate, in which the heart rate shifts towards and/or substantially synchronizes with the frequency of the pressure pulse. The range within which a patient's heart rate may be manipulated depends on physiological limitations of the patient, as well as the degree with which the particular patient responds to the pressure pulse. In this way, a desired therapeutic effect may include inducing a desired heart rate during thermal contrast therapy.

In addition to peristaltic pumps, other means for transferring fluid from the reservoir to the one or more treatment cuffs, and/or other means for generating pressure pulses are within the scope of the present disclosure.

### Temperature Control

Referring to FIG. 2A, a thermal contrast therapy device may be equipped with a hot reservoir 200 and a cold reservoir 202. The hot reservoir is configured to store a volume of hot heat transfer fluid for use primarily during heating periods, and the cold reservoir is configured to store a volume of cold heat transfer fluid for use primarily during cooling periods. Hot heat transfer fluid is pumped from the hot reservoir to a treatment cuff during heating periods, and cold heat transfer fluid is pumped from the cold reservoir to a treatment cuff during cooling periods.

Hot heat transfer fluid may be heated via a heating element 204 to a temperature sufficient to provide fluid of a desired temperature for heating periods. Heating may be accomplished by any means known in the art, for example using a cartridge heater mounted within the hot reservoir. Hot heat transfer fluid may be heated to a range of temperatures based on the desired temperature of the heat transfer fluid to be supplied to the treatment cuff.

Similarly, cold heat transfer fluid may be cooled via a cooling module 206 to a temperature sufficient to provide fluid of a desired temperature for cooling periods. Cooling may be accomplished by any means known in the art, for example, using compressor/refrigerant systems, heat exchanger, or thermoelectric cooling technology such as a Peltier cooler. Cold heat transfer fluid may be cooled to a range of temperatures based on the desired temperature of the heat transfer fluid to be supplied to the treatment cuff.

In some embodiments, a thermal contrast therapy device may be equipped with one or more temperature sensors. For example, as depicted in FIG. 2A, temperature sensors 220 and 222 are positioned at the inflow and/or outflow lines to and from the treatment cuff, and temperature sensors 224 and 226 are positioned at the cold reservoir and the hot reservoir, respectively. As depicted in FIG. 2B, one or more temperature sensors 238 may be positioned at various locations on or within the treatment cuff 210. Temperature sensor(s) 238 may be configured to measure the temperature of the patient's tissue proximate to the treatment cuff. Additionally and/or in the alternative, temperature sensor(s) 238 may be configured to measure the temperature of heat transfer fluid at a given point within the treatment cuff.

Processor 218 may be configured to control the temperature of the heat transfer fluid, utilizing temperature measurements from any one or more temperature sensors. For example, temperature sensors 220 and/or 222 may be used to control the temperature of the heat transfer fluid based on the temperature of the fluid entering or exiting the treatment cuff, respectively. Temperature sensor(s) 238 may be configured to control the temperature of the heat transfer fluid based on the temperature of the patient's tissue proximate to the treatment cuff and/or the temperature of the fluid at a given point within the treatment cuff. Such temperature control may be configured to enable the device to provide customized thermal contrast therapy treatment sequences based, at least in part, on fluid temperature in accordance with the present disclosure.

Customized thermal contrast therapy treatment sequence may be provided, for example, by varying the temperature of the heat transfer fluid during heating periods and/or cooling periods, and providing fluid of a desired temperature to a treatment cuff. In some embodiments, the temperature of the heat transfer fluid in the hot reservoir and/or the cold reservoir may be increased or decreased during the course of a thermal contrast therapy treatment so as to provide heat transfer fluid having a desired temperature during the various heating periods and/or cooling periods. In other embodiments, the respective temperatures of the hot heat transfer fluid and cold heat transfer fluid may be maintained relatively constant, and the two fluids may be used in combination to temper one another in various proportions. Such tempering allows a thermal contrast therapy device to provide a range of desired fluid temperatures depending on the degree of tempering, without having to repeatedly heat or cool a reservoir of heat transfer fluid to the desired temperature. Thus, during a heating period, hot heat transfer fluid may be tempered with a measure of cold heat transfer fluid via control valve(s) 212 so as to attain a desired fluid temperature at the treatment cuff. Similarly, during a cooling period, cold heat transfer fluid may be tempered via control valve(s) 212 with an amount of hot heat transfer fluid.

In some embodiments, as depicted in FIG. 2B, a thermal contrast therapy device may be equipped with a neutral reservoir 232 configured to store neutral-temperature heat transfer fluid. The neutral-temperature heat transfer fluid may be used to provide neutral temperature periods, or to temper hot heat transfer fluid and/or cold heat transfer fluid during respective heating periods and/or cooling periods. Heat transfer fluid in the neutral reservoir may be maintained at a range of appropriate neutral temperatures. Additionally and/or in the alternative, hot heat transfer fluid may be mixed with cold heat transfer fluid to provide a desired temperature for the neutral-temperature fluid.

Heat transfer fluid exiting a treatment cuff may be returned to any one or more of the reservoirs, as depicted in FIG. 2B, via one or more fluid return control valves 216. In some embodiments, fluid returning from the treatment cuff may be directed to a given reservoir based on the temperature of the fluid, for example, so as to optimize the heating and cooling efficiency of the thermal contrast therapy device. Alternatively, fluid returning from the treatment cuff may be routed to a neutral reservoir 232, for example, so that mixing of fluids from heating periods and cooling periods may provide a fluid temperature approximately close to the desired temperature for neutral treatments. Additionally, neutral-temperature heat transfer fluid may be adjusted by tempering with hot or cold heat transfer fluid, as appropriate. For example, as depicted in FIG. 2B, the temperature of the fluid in the neutral reservoir may be adjusted with fluid from the hot reservoir and/or the cold reservoir using a circulation pump 234 and one or more circulation valves 236 configured to transfer fluid from the hot reservoir and/or the cold reservoir to the neutral reservoir. Alternatively, neutral-temperature heat transfer fluid may be tempered with hot or cold heat transfer fluid via one or more feed control valves 212.

Heat transfer fluids may comprise any fluid or combination of fluids known in the art for effecting heat transfer. Exemplary fluids include water, propylene glycol, saline, or combinations thereof.

### Flow Control

In some embodiments, a thermal contrast therapy device may be equipped with one or more flow meters. For example, as depicted in FIG. 2A, flow meter 228 may be positioned at the inflow line to the treatment cuff. Processor 218 may be configured to control the flow rate of the heat transfer fluid, utilizing flow measurements from any one or more flow meters. The flow rate of the heat transfer fluid may be modified, for example, by varying the speed of one or more feed pumps 208 and/or by varying the positioning of one or more flow control valves/flow regulators 214. Such flow control may be configured to enable the device to provide customized thermal contrast therapy treatment sequences based, at least in part, on fluid flow rate in accordance with the present disclosure. Additionally, in some embodiments customized thermal contrast therapy treatment sequences may be based, at least in part, on both flow rate and temperature of the heat transfer fluid.

The driving force for heat transfer during thermal contrast therapy is the difference in temperature between the patient's body and the heat transfer fluid. As heat transfer occurs between the heat transfer fluid and the area of the patient's body proximate to the treatment cuff, the temperature of the heat transfer fluid within the treatment cuff will shift towards the temperature of the patient's body. The magnitude of this change in temperature is inversely proportional to the flow rate of the heat transfer fluid. A relatively high flow rate will yield a relatively low change in temperature between the inflow and outflow ports of the treatment cuff bladder, and thus a relatively high driving force for heat transfer. Conversely, a relatively low flow rate will yield a relatively high change in temperature between the inflow and outflow ports of the treatment cuff bladder, and thus a relatively low driving force for heat transfer. Thus, the rate of heat transfer effected by the heat transfer fluid depends on flow rate, and the rate of heat transfer effected during heating periods and/or cooling periods may be controlled by varying the flow rate of the heat transfer fluid. Accordingly, at a constant fluid temperature entering the treatment cuff, the rate of heat transfer may be increased by increasing the flow rate of the heat transfer fluid, to the point where the change in temperature of the heat transfer fluid between the inflow port and the outflow port of the treatment cuff approaches zero. Conversely, the rate of heat transfer may be decreased by decreasing the flow rate of the heat transfer fluid, to the point where the difference in temperature between the patient's body and the heat transfer fluid approached zero.

Thus, customized thermal contrast therapy treatment sequences may be provided in accordance with the present disclosure which are based, at least in part, on providing a specified flow rate of the heat transfer fluid. For example, a customized thermal contrast therapy treatment sequence which calls for effecting a desired measure of heat transfer may be provided by varying the flow rate of the heat transfer fluid. In some embodiments, a desired rate of heat transfer during heating periods and/or cooling periods may be effected solely by varying the flow rate of the respective hot heat transfer fluid and/or cold heat transfer fluid. Alternatively, the desired measure of heat transfer may be attained by varying both the flow rate and the temperature of the heat transfer fluid. For example, in some embodiments, the temperature of the heat transfer fluid may be varied to attain a close approximation of the desired rate of heat transfer, and then the flow rate of the heat transfer fluid may be varied to provide fine-tuning to more precisely control the rate of heat transfer.

In some situations, thermal contrast therapy may be provided under circumstances where a patient may be particularly sensitive to hot and/or cold heat transfer fluid temperatures. This may be the case, for example, with patients suffering from frostbite or hypothermia, elderly patients, patients with diabetes and related physiological complications (e.g., gangrene), patients with lymphedema or other disorders associated with vascular or lymphatic insufficiency (e.g., Chronic Venous Insufficiency, venous stasis ulcers, post-mastectomy edema or chronic lymphedema), patients with peripheral vascular disease or other circulatory deficiency syndrome (e.g., arteriosclerosis, deep vein thrombosis, Buereger's disease, or thromboangiitis obliterans). Such sensitive patients may be unable to tolerate certain fluid temperature levels without severe discomfort or injury to tissue. In these instances, providing heat transfer fluid of a given temperature may not be a viable means for effecting a desired rate of heat transfer because the fluid temperature may exceed the patient's pain tolerance and/or cause injury to tissue. Instead, the rate of heat transfer may be increased by increasing the flow rate of the heat transfer fluid, while maintaining a more moderate fluid temperature which is within the patient's pain tolerance and/or which will not cause injury to tissue. Thus, at a given fluid temperature which is tolerable to a sensitive patient, an enhanced rate of heat transfer may be provided by increasing the flow rate of the heat transfer fluid. This enables the induction of enhanced cycles of vasoconstriction and vasodilation, enhanced improvements in blood flow, and other enhanced therapeutic effects, while maintaining a moderate fluid temperature that is suitable for sensitive patients.

During a thermal contrast therapy treatment sequence, the temperature gradient between the heat transfer fluid entering the treatment cuff bladder and the patient's tissue proximate to the treatment cuff may decrease as a result of the ensuing heat transfer. Thus, at a constant flow rate and temperature of the heat transfer fluid entering the treatment cuff bladder, the rate of heat transfer may decline over the course of a heating period or cooling period due to such decreasing temperature gradient. In some embodiments, a customized thermal contrast therapy treatment sequence may be provided in which the flow rate of the heat transfer fluid is adjusted so as to maintain a desired rate of heat transfer notwithstanding changes in the temperature of the tissue proximate to the treatment cuff. Such changes in flow rate may be effected, for example, over the course of a given heating period and/or cooling period, and/or from one period to the next over the course of the treatment sequence. For example, as the tissue proximate to the treatment cuff changes temperature in response to heat transfer, the flow rate of the heat transfer fluid may be increased so as to offset the decreasing rate of heat transfer that would otherwise result as tissue temperature proximate to the treatment cuff shifts towards the temperature of the heat transfer fluid entering the treatment cuff.

Additionally, temperature changes to a patient's tissue effected during one heating period or cooling period may impact the rate of heat transfer in a subsequent period. For example, alternating heating periods and cooling periods may provide a greater temperature gradient between the patient's tissue and the heat transfer fluid in subsequent alternating periods. Thus, the driving force for heat transfer in a subsequent period may be enhanced, at least initially, due to the change in tissue temperature effected by the preceding period. Such enhanced temperature gradient may be desirable or undesirable, depending on the desired effects of the particular thermal contras therapy treatment sequence. In some embodiments, the flow rate of the heat transfer fluid may be modified based, at least in part, on the temperature gradient between the patient's tissue and the heat transfer fluid. This may include effecting a change in the flow rate of the heat transfer fluid in consideration of an enhanced temperature gradient effected by one or more preceding periods. In some embodiments, a sequence of alternating cooling periods and heating periods may be configured to provide an enhanced temperature gradient in one or more subsequent periods within the sequence. In some embodiments, a neutral period may be provided between one or more of the alternating cooling periods and heating periods so as to provide a subdued temperature gradient in one or more subsequent periods within the sequence, for example, the period following the neutral period. In some embodiments, the flow rate of the heat transfer fluid may be varied to effect a desired temperature gradient during at least a portion of a heating period and/or cooling period.

In some embodiments, the artisan may derive advantages from utilizing changes in flow rate, in addition to or as an alternative to changes in fluid temperature, to effect a desired rate of heat transfer. In some embodiments, the heating and cooling loads on a thermal contrast therapy device may be reduced by using flow rate to effect a desired rate of heat transfer. This may yield more efficient, cost-effective devices, as well as enhanced ability to provide customized treatment sequences in accordance with the present disclosure. Additionally, the rate of heat transfer may, in some embodiments, be more effectively controlled via flow rate of the heat transfer fluid than via the temperature of the heat transfer fluid. This may be the case, for example, regarding embodiments having configurations such that the rate of heat transfer may be changed more rapidly and/or more precisely via a change in flow rate than via a change in temperature. In certain embodiments where flow rate control facilitates more precise and/or more rapid control over the rate of heat transfer, customized thermal contrast therapy treatment sequences may be provided based, at least in part, on flow rate control. Some such sequences may not be feasibly attained in certain embodiments except via flow rate control. For example, a customized treatment sequences may include rapidly transitioning between heating periods and cooling periods, rapidly changing the rate of heat transfer, and/or more precisely controlling the rate of heat transfer. These rapid changes may be feasible via flow rate control but not via temperature control in certain embodiments.

Additionally, in some embodiments, the artisan may desire to minimize or eliminate the transition times between heating periods and cooling periods. To accomplish this, treatment cuffs may be provided which are equipped with a bladder having a relatively small fluid capacity so as to enable heat transfer fluid to be more rapidly flushed from the bladder. A small fluid capacity may enhance the effect of flow rate on the rate of heat transfer, and consequently the range of control over the rate of heat transfer via flow rate. Accordingly, in some such embodiments, the rate of heat transfer may be more rapidly changed via a change in the flow rate of the heat transfer fluid than via a change in the temperature of the heat transfer fluid.

### Calibration

In some embodiments, a thermal contrast therapy device may be calibrated based on variations in the device and/or the preferences of one or more users of the device. A given treatment sequence may yield different effects due to differences between various devices (e.g., different manufacturers or model numbers), variations between devices (e.g., manufacturing differences between production runs), or set-up configurations (e.g., the length of the inflow and/or outflow lines between the device and treatment cuff). These differences may be compensated for by calibrating treatment sequences for a particular device. A thermal contrast therapy device may be calibrated, for example, by measuring fluid temperature and/or flow rate, etc. with calibration equipment and adjusting calibration settings for the device. Calibration allows for customized thermal contrast therapy treatment sequences to be provided with consistency across a plurality of devices and/or for a plurality of users.

The artisan will appreciate that a given treatment sequence may yield different effects on different patients due to differences between patients, such as physiological attributes or personal preference. These differences may similarly be compensated for by providing a calibration adjustment corresponding to a particular patient. For example, a treatment sequence may be calibrated for a particular patient by providing a calibration adjustment to any one or more treatment sequence parameters. Providing a calibration adjustment may include, for example, shifting a set-point corresponding to a sequence parameter by a factor. As examples, the set-point for all heat transfer fluid temperatures in a treatment sequence may be shifted up or down; the set-point for all desired measures of heat transfer (i.e., rate of heat transfer or quantity of heat transfer) in a treatment sequence may be shifted up or down; or the number of periods in a treatment sequences and/or the duration of a treatment sequences may be shifted up or down. The calibration adjustment and/or factor may be based on a percentage, an absolute value, a formula, or the like. The calibration adjustment and/or factor may be derived empirically for an individual patient, and/or from data in a database associated with a thermal contrast therapy system, for example, data that provides a correlation to treatment sequences associated with other users. Such calibration adjustments may be applied to all treatment sequences, a subset of treatment sequences, or to a particular device, etc., and/or any of the foregoing as associated with a particular user.

### III. Methods for Providing Thermal Contrast Therapy

In accordance with the present disclosure, various methods are described herein for providing thermal contrast therapy treatments using the systems and devices described herein. The methods disclosed herein include computer-implemented methods of providing thermal contrast therapy.

Thermal contrast therapy comprises circulating heat transfer fluid from a thermal contrast therapy device through a treatment cuff, the treatment cuff having been applied to a patient, while providing a sequence comprising a plurality of alternating cooling periods and heating periods. In some embodiments, a treatment sequence may include a plurality of transition periods, each occurring between alternating cooling periods and heating periods. During such transition periods, the rate of heat transfer between the patient's tissue proximate to the treatment cuff and the heat transfer fluid is transitioned from a first heat transfer rate which corresponds to the period preceding the transition period to a second heat transfer rate which corresponds to the period following the transition period. Changes to the rate of heat transfer may be effected by varying the flow rate and/or temperature of the heat transfer fluid. The flow rate and/or fluid temperature may be changed according to any desirable transition curve, and may also include providing a period of neutral-temperature heat transfer fluid and/or neutral rate of heat transfer.

In some embodiments, a customized thermal contrast therapy treatment sequence is provided. A customized treatment sequence may correspond to a treatment program having been prescribed to a user, or selected by a user of a thermal contrast therapy device. The customized treatment sequence may be based upon, for example, conditions particular to the patient intended to receive treatment, a physiological parameter value exhibited by the patient, the particular area of the body sought to be treated, the particular symptom or condition sought to be treated, the particular desired therapeutic result, and/or other considerations. In some embodiments, a customized treatment sequence may be uploaded or stored in a database accessible by a thermal contrast therapy system or device. For example, a treatment provider or a user may select a treatment from a menu of one or more treatment options in a database associated with a thermal contrast therapy system configured to provide customized thermal contrast therapy treatment programs. Alternatively, a customized treatment sequence may be manually input by the treatment provider or user.

It is believed that a patient's response to thermal contrast therapy depends on a number of variables related to the conditions of the treatment sequence, including the rate of heat transfer between the heat transfer fluid and the patient's tissue proximate to the treatment cuff, the temperature of the heating periods and/or cooling periods, the pressure applied during treatment; the quantity and sequence of the heating periods and/or cooling periods, the various cycle times for each heating period and/or cooling period; the duration and rate of change of transitions between heating periods and/or cooling periods, including whether a neutral temperature period is provided between heating periods and/or cooling periods, the duration of the thermal contrast therapy treatment sequence, the frequency with which thermal contrast therapy treatment is to be provided, the dimensions and configuration of the treatment cuffs, the pulsation frequency of the heat transfer fluid, and other variables. Any one or more of these variables may, in accordance with the present disclosure, form the basis of a customized thermal contrast therapy treatment sequence, for example, to provide a treatment sequence or series of treatments optimized to enhance one or more therapeutic effects of thermal contrast therapy.

A patient's response to thermal contrast therapy may additionally depend on certain characteristics particular to the patient, including, for example, the patient's age, gender, body mass, fat content, physical fitness, energy level, and general state of health. Physiological characteristics as well as thermodynamic properties may also impact a patient's response to thermal contrast therapy. This includes a patient's ability to generate body heat, ability to repair the loss of heat, and to support the loss of heat without negatively impacting the body's vital processes, as well as the condition of the patient's nervous system at the time of treatment, and the extent to which the patient is accustomed to thermal contrast therapy.

Other variables which may affect a patient's response to thermal contrast therapy include factors related to any particular chronic or acute condition or ailment affecting the patient, including a condition or ailment sought to be treated with thermal contrast therapy. Thermal contrast therapy treatment sequences, as well as frequencies between treatments, may be provided which depend on these and other characteristics particular to the patient intended to be treated. Thermal contrast therapy procedures may be formulated or provided which depend on the particular body part(s) sought to be treated, particular conditions or symptoms sought to be treated, and/or the desired therapeutic result sought to be attained from the treatment. Exemplary treatments, procedures, and sequences are described in detail herein.

### Heat Transfer

A thermal contrast therapy treatment may be quantified based on measures of heat transfer, for example, the rate of heat transfer and/or the quantity of heat transfer effected during the respective heating periods and cooling periods of a treatment sequence. Without wishing to be bound by theory, it is believed that various therapeutic effects of thermal contrast therapy may be enhanced by providing customized treatments which effect a particular measure of heat transfer (i.e., a particular rate of heat transfer and/or quantity of heat transfer). In some embodiments, a customized thermal contrast therapy treatment sequence includes effecting a desired measure of heat transfer between the fluid and the patient's tissue proximate to the treatment cuff during one or more of the cooling periods and heating periods. The desired measure of heat transfer may be effected by automatically adjusting the flow rate and/or temperature of the heat transfer fluid.

The rate of heat transfer is expressed as:

$$q = \frac{m}{t} {c}_{p} Δ T \qquad ( 1 )$$

where q is the mean heat transfer rate,

$$\frac{m}{t}$$

is the mass flow rate per unit time, cₚ is the specific heat capacity of the fluid, and ΔT is the temperature gradient. Those skilled in the art will appreciate that q represents an optimum heat transfer rate, and that in practice, while the vast majority of heat transfer during a during thermal contrast therapy treatment will be between the heat transfer fluid and the patient's tissue proximate to the treatment cuff, a portion of the heat transferred will be attributable to other considerations. For example, a portion of the heat will transfer to the cuff material and/or the surrounding atmosphere, etc. These considerations may be quantified empirically and/or by principles of thermodynamics known in the art, and the mean heat transfer rate during a given heating period or cooling period within a thermal contrast therapy treatment sequence may thus be expressed as:

$${q}_{TCT} = k \frac{m}{t} {c}_{p} Δ T \qquad ( 2 )$$

where k is an efficiency constant which represents the proportion of heat transferred to the tissue proximate to the treatment cuff.

The total heat transferred during a given heating period or cooling period may be obtained from:

Q_{TCT}=q_{TCT}Δt (3)

where Δt is the duration of the applicable heating period or cooling period. Likewise, the heat transferred from the various heating periods and/or cooling periods may be summed to provide the total heat transferred during n periods, where n represents all or a subset of the periods comprising a thermal contrast therapy treatment sequence, as follows:

Q_{TCT}=Σₙ₌₁ⁿq_{TCT}_{₁}Δt₁+q_{TCT}_{₂}Δt₂+ . . . +q_{TCT}_{ₙ}Δtₙ (4)

In some embodiments, it may be advantageous to sum the heating periods and cooling periods separately, so as to ascertain the total heat transferred to the patient during heating periods and the total heat transferred from the patient during cooling periods.

In some embodiments it may be advantageous to model the heating and/or cooling of a patient's tissue during thermal contrast therapy. Such models may be used to evaluate treatment outcomes relative to model predictions, to perform research on various thermal contrast therapy treatment sequences and parameters thereof, and/or to suggest and evaluate new treatment strategies, etc.

The general factors to consider when quantifying heat transfer during thermal contrast therapy include heat transfer effected during the treatment, heat production due to metabolic processes, heat transfer due to blood perfusion, geometry of the thermal contrast therapy treatment area (e.g., as defined by the one or more treatment cuffs), the thermophysical properties of various types of body tissue, and thermoregulatory mechanisms.

Across a control volume, the principle of conservation of energy provides that the balance of thermal energy can be states as:

q_{TCT}=q_{storage}+qₗₒₛₛ+qₘₑₜ+W (5)

where q_{TCT} is the heat energy gained by the control volume, for example from a given heating period or cooling period during a thermal contrast therapy treatment, q_{storage} is the heat energy stored within the control volume of tissue and fluid, qₗₒₛₛ is the heat energy lost through the boundary of the control volume, qₘₑₜ is the heat energy produced by metabolic heating, and W is work performed on the control volume.

Heat is transferred by conduction, convection, evaporation, and radiation. The two primary mechanisms for heat transfer inside tissue during thermal contrast therapy are convection and conduction. Under most conditions for thermal contrast therapy, heat transfer by evaporation (or perspiration) and radiation, as well as the work, W, are negligible. The temperature gradient inside the tissue drives heat transfer through conduction, and blood perfusion drives heat transfer through convection.

According to Fourier's law, the conductive heat transfer between two layers is found by the relation:

$${q}_{cond} = - kA \frac{dT}{dx} \qquad ( 6 )$$

where q_{cond} is the conductive heat transfer per unit time, k is the thermal conductivity, A is the cross sectional area and dT/dx is the temperature gradient in the direction of heat transfer across a material of thickness x.

Those skilled in the art will appreciate that several approaches exist for modeling heat transfer from blood perfusion and that any one or more of such models may be used in accordance with the present disclosure. Approaches for modeling heat transfer from blood profusion include: the Pennes bioheat model; the Wulff continuum model; the Klinger continuum model; the Chen-Holmes (CH) continuum model; the Weinbaum, Jiji and Lemons (WJL) bioheat model; the Simplified Weinbaum-Jiji model; the Zolfaghari and Maerefat simplified thermoregulatory bioheat model. These models generally employ a certain level of approximation, particularly because blood circulates in a variety of vessels ranging in lumen diameter from the approximately 2.5 cm, in the case of the aorta, to the approximately 6-10 μm, in the case of capillaries. More precise models based on computer algorithms or empirical research may also be utilized in accordance with the present disclosure.

These aforementioned blood perfusion models generally rely on the basis of one of two main approaches: the continuum approach and the discrete vessel (vascular) approach. In the continuum approach, the thermal impact of all blood vessels are modeled with a single global parameter; and in the vascular approach, the impact of each vessel is modeled individually. The Pennes bioheat model is one of the most widely used approaches for modeling heat transfer from blood perfusion. The Pennes bioheat model assumes that the rate of heat transferred by the circulating blood at the capillary level equals the difference between the venous and arterial temperatures multiplied by the flow rate, as follows:

q_{blood}=ρ_{b}c_{b}w_{b}(Tₐ−Tᵥ) (7)

where ρ_{b} is the density of the patient's blood, c_{b} is the specific heat of the patient's blood, w_{b} is the perfusion rate, Tₐ is the arterial blood temperature, and Tᵥ is the venous blood temperature.

The heat energy stored in the control volume can be expressed as follows:

$${q}_{storage} = {\int }_{v} ρ c ( \overline{x} ) \frac{dT ( \overline{x} , t )}{dt} dv \qquad ( 8 )$$

where ρ is the tissue density, c is the specific heat, and T is the tissue temperature.

The terms of equations (6), (7), and (8) may be substituted into equation (5) and integrated over the entire volume and surface area, to obtain the Pennes bioheat equation:

$$ρ c \frac{\partial T}{\partial t} = ∇ k ∇ T + {ρ}_{b} {c}_{b} {w}_{b} ( {T}_{a} - {T}_{v} ) + {q}_{met} \qquad ( 9 )$$

The Pennes bioheat equation can thus be used to analyze heat transfer in various body tissues under various thermal contrast therapy treatment sequences, including the customized treatment sequences disclosed herein.

The Pennes bioheat model assumes that the blood perfusion effect is homogenous and isotropic and that thermal equilibration occurs in the microcirculatory capillary bed due to the low blood flow velocity at the capillary level. Thus, the Pennes bioheat model assumes that blood enters capillaries at the temperature of arterial blood, Tₐ, where convective heat transfer occurs to bring the temperature to that of the surrounding tissue, such that the temperature at which the blood enters the venous circulation is that of the local tissue.

Pennes performed a series of studies to validate this model, which show reasonable agreement between the model and experimental data. Those skilled in the art will appreciate that while the Pennes bioheat model is often adequate, there are certain shortcomings in this model due to its inherent simplicity, and that other models, for example, those which utilize the discrete vessel (vascular) approach, may be more suitable in situations where enhanced precision is desired. For example, the Pennes bioheat model does not account for countercurrent heat transfer between adjacent vessels, or directionality effects due to the presence of larger blood vessels, or heat exchange with larger vessels in which complete thermal equilibrium may not be assumed. The CH continuum model and the WJL bioheat model, for example, address these issues. Accordingly, such other models may be used to analyze heat transfer as an alternative or in addition to the Pennes bioheat model.

Conditions particular to the patient intended to receive treatment and/or the particular area of the body sought to be treated, among other factors, may cause a given thermal contrast therapy treatment sequence to effect a different measure of heat transfer (i.e., a different rate of heat transfer and/or a different quantity of heat transfer). For example, various thermodynamic properties and other factors affecting heat transfer may be attributable to a patient's age, gender, body mass, body fat percentage, body mass index, metabolic rate, neural sensitivity, physical fitness, energy level, and general state of health. This includes the thermal conductivity, density, and heat capacity of a patient's body tissues and fluids, the surface thickness of various layers (e.g., dermis, epidermis, fat, muscle) of a patient's body tissue, factors related to a patient's blood perfusion, including blood pressure, blood flow rate, venous structure (e.g., capillary density, lumen diameter distribution, etc.), and a patient's metabolic rate. Likewise, given the various factors which may impact the measure of heat transfer effected during a given thermal contrast therapy treatment sequence, as between two patients and/or body parts sought to be treated, a different treatment may be required for each in order to effect the same measure of heat transfer during the respective treatments. Furthermore, different thermal contrast therapy treatment sequences may be required for the same patient as, when, and if factors change which may impact the measure of heat transfer effected during a given treatment sequence.

Customized thermal contrast therapy treatment sequences may be provided in accordance with the present disclosure which comprise effect a desired measure of heat transfer during the treatment sequence and/or the one or more of the heating periods and/or cooling periods thereof. The desired measure of heat transfer (i.e., rate of heat transfer and/or quantity of heat transfer) may depend on any one or more of the factors affecting thermal contrast therapy treatments such as the factors disclosed herein, including: conditions particular to the patient intended to receive treatment, the particular area of the body sought to be treated, the particular symptom or condition sought to be treated, the particular desired therapeutic result, and/or other considerations.

### Relationship Between Flow Rate and Heat Transfer

The rate of heat transfer is directly proportional to flow rate. Increasing flow rate increases the rate of heat transfer and decreasing flow rate decreases the rate of heat transfer.

The following example illustrates the relationship between the flow rate of heat transfer fluid and the rate of heat transfer effected by the heat transfer fluid. A cooling period was provided, via a thermal contrast therapy device having a treatment cuff attached to a patient's lower leg. The temperature of the heat transfer fluid (water) entering the treatment cuff was maintained at about 40° F. At a flow rate of about 120 mL per minute, the heat transfer fluid exiting the treatment cuff was about 58° F. At a flow rate of about 240 mL per minute, the heat transfer fluid exiting the treatment cuff was about 53° F. Thus, using equation (1), the rate of heat transfer to the fluid was about 4.7 BTU per minute when the flow rate was 120 mL per minute (120 mL/min×1 sq. ft/28,316.8 mL×62.24 lb/sq. ft×1 BTU/lb-° F.×18° F.), and about 6.9 BTU per minutes when the flow rate was 240 mL per minute (240 mL/min×1 sq. ft/28,316.8 mL×62.24 lb/sq. ft×1 BTU/lb-° F.×13° F.).

### Dynamically Controlled Treatment Sequences

A customized thermal contrast therapy treatment sequence may include dynamically controlling the sequence to effect a desired treatment and/or to attain a desired therapeutic effect. Any one or more variables of the treatment sequence may be dynamically controlled in order to provide a customized treatment sequence in accordance with the present disclosure. In some embodiments, automatic adjustments to a thermal contrast therapy treatment sequence may include: changing a setting for the temperature and/or flow rate of the heat transfer fluid; changing the duration of one or more of the cooling periods, heating periods, and/or transition periods; changing the number of cooling periods, heating periods, and/or transition periods; changing the rate of change of the temperature and/or flow rate of the heat transfer fluid; and/or prescribing a sequence for one or more future thermal contrast therapy treatments.

In some embodiments, the treatment sequence may be dynamically controlled to provide a desired measure of heat transfer (i.e., a rate of heat transfer and/or quantity of heat transfer) during one or more of the heating periods and/or cooling periods. The measure of heat transfer during one or more of the heating periods and/or cooling periods may be controlled, for example, using equations (2) and/or (3) and by adjusting the flow rate and/or temperature of the heat transfer fluid, and/or the duration of the one or more periods. The rate of heat transfer, q_{TCT}, may be controlled for a given period, for example, using equation (2) and then adjusting the temperature gradient between the heat transfer fluid and the patient's tissue proximate to the treatment cuff, ΔT. The quantity of heat transfer, Q_{TCT}, may be controlled, for example, using equation (3) and then adjusting the rate of heat transfer, q_{TCT} and/or the duration of the period, Δt. The temperature gradient may be controlled by adjusting the temperature and/or the flow rate of the heat transfer fluid. Dynamically controlled thermal contrast therapy treatment sequences may be provided which utilize any one or more equations for modeling blood perfusion (e.g., the Pennes bioheat model), and/or which take into account other factors which may impact heat transfer during thermal contrast therapy. Such factors include heat production due to metabolic processes, geometry of the thermal contrast therapy treatment area (e.g., as defined by the treatment cuffs), the thermophysical properties of various types of body tissue, and/or thermoregulatory mechanisms.

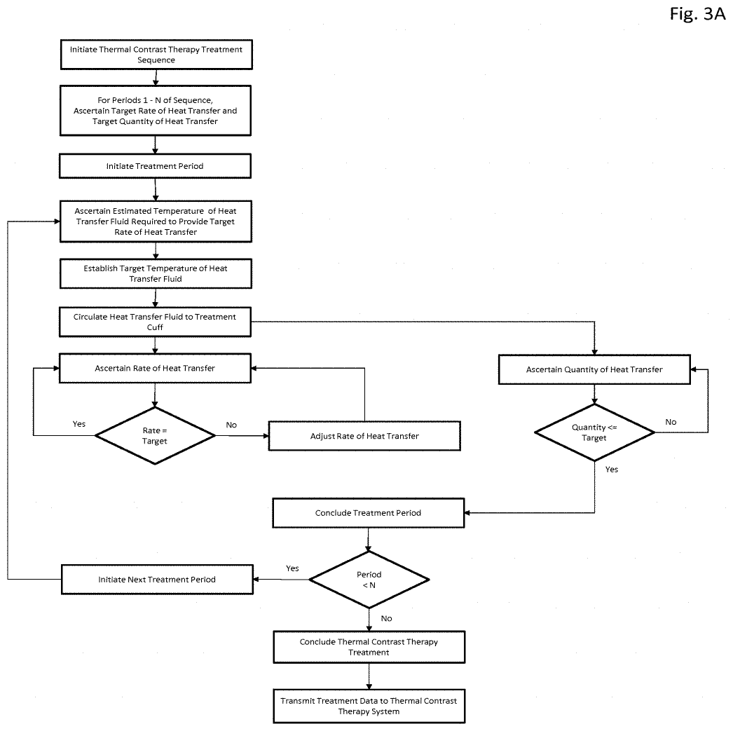

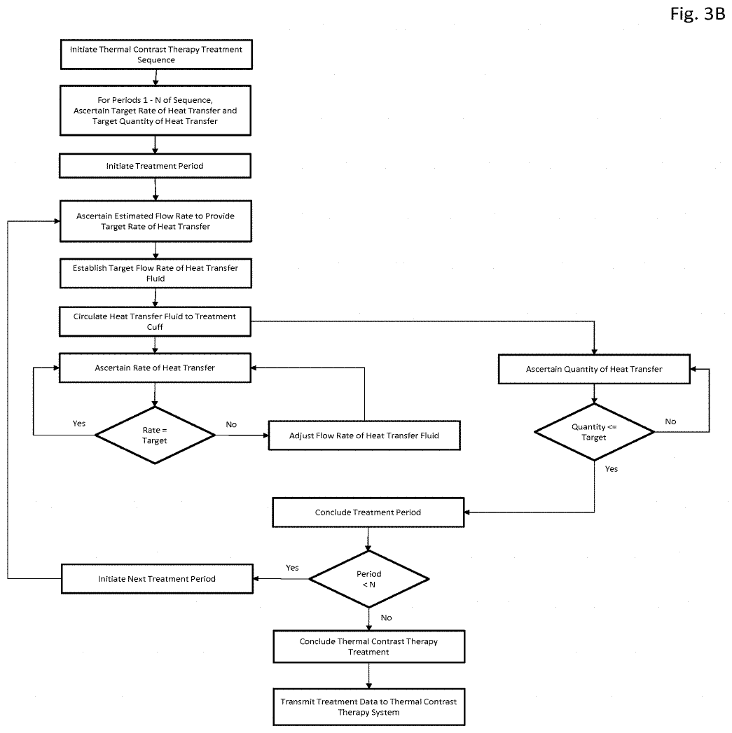

Referring to FIG. 3A, a flow chart illustrative of an exemplary thermal contrast therapy treatment process for providing a desired measure of heat transfer is shown. The thermal contrast therapy device initiates the thermal contrast therapy treatment sequence. The sequence may be any sequence in accordance with the present disclosure. For each period of the treatment sequence, heat transfer fluid is circulated to the treatment cuff, the temperature of the heat transfer fluid being approximately that which is required to provide the desired rate of heat transfer. Throughout the course of the treatment period, the device ascertains the rate of heat transfer and determines whether the actual rate of heat transfer matches the target rate of heat transfer. The target may be a specific value or a range. If the rate of heat transfer does not match the target rate, then the rate of heat transfer is adjusted. The rate of heat transfer may be adjusted by changing the temperature and/or flow rate of the heat transfer fluid in accordance with the present disclosure.

Throughout the course of each treatment period within the sequence, the thermal contrast therapy device ascertains the quantity of heat transfer effected and determines whether the actual quantity of heat transfer is less than or equal to the target quantity. In some embodiments, the period is concluded when the quantity of heat transfer reaches or exceeds the target quantity. Alternatively, the period may continue for a specified duration of time and conclude when the specified duration has elapsed.

When the treatment period has concluded, the thermal contrast therapy device ascertains whether there are additional periods remaining in the sequence. If there are additional periods remaining, the device proceeds through such remaining periods until all of the periods have been administered. The treatment sequence concludes when all of the periods have been administered, at which point data associated with the treatment sequence maybe transmitted to a thermal contrast therapy system for use in accordance with the present disclosure.

Referring to FIG. 3B, a flow chart illustrative of an exemplary thermal contrast therapy treatment process for providing a desired measure of heat transfer, utilizing flow rate to modify the rate of heat transfer, is shown. The sequence depicted in FIG. 3B proceeds similar to the sequence depicted in FIG. 3A, except that the rate of heat transfer is controlled entirely by the flow rate of the heat transfer fluid.

In some embodiments, a thermal contrast therapy treatment sequence may include rapidly alternating cooling periods and heating periods. Such a sequence may be effective to induce cycles of vasoconstriction and vasodilation. Cycles of vasoconstriction and vasodilation may be provided in a manner effective to cause an increase in blood circulation and/or blood oxygen content in a patient's tissue proximate to the treatment cuff during thermal contrast therapy.

In some embodiments, a customized thermal contrast therapy treatment sequence may be dynamically controlled to optimize one or more physiological parameter values. For example, one or more variables of a treatment sequence may be automatically adjusted when the patient exhibits a physiological parameter that corresponds to a predefined value. The physiological parameter values may be based on the patient's vital signs. A predefined value may include, for example, an increase from a previous value, a decrease from a previous value, a previous value, and/or a target value.

Physiological parameters which may be monitored and utilized to dynamically control a thermal contrast therapy treatment sequence include: body temperature (e.g. core temperature, local temperature, etc.), heart rate, blood pressure, blood flow, blood oxygen level, or other vital signs. Such vital signs may be monitored using any number of devices which are well known in the art. For example, a thermal contrast therapy treatment sequences may be automatically adjusted so as to optimize the magnitude of an increase in blood circulation and/or blood oxygen content induced by cycles of vasoconstriction and vasodilation.

In some embodiments, a treatment sequence may be based on a physiological parameter value exhibited by a patient during treatment. For example, based on a patient's vital signs, adjustments may be made during treatment to any one or more of: the fluid temperature provided during heating periods and/or cooling periods, the duration and/or number of heating periods and/or cooling periods, the quantity of heat transfer and/or rate of heat transfer during one or more of the heating periods and/or cooling periods. A treatment sequence may also be based on a physiological parameter value exhibited by a patient prior to a treatment or during a previous treatment.

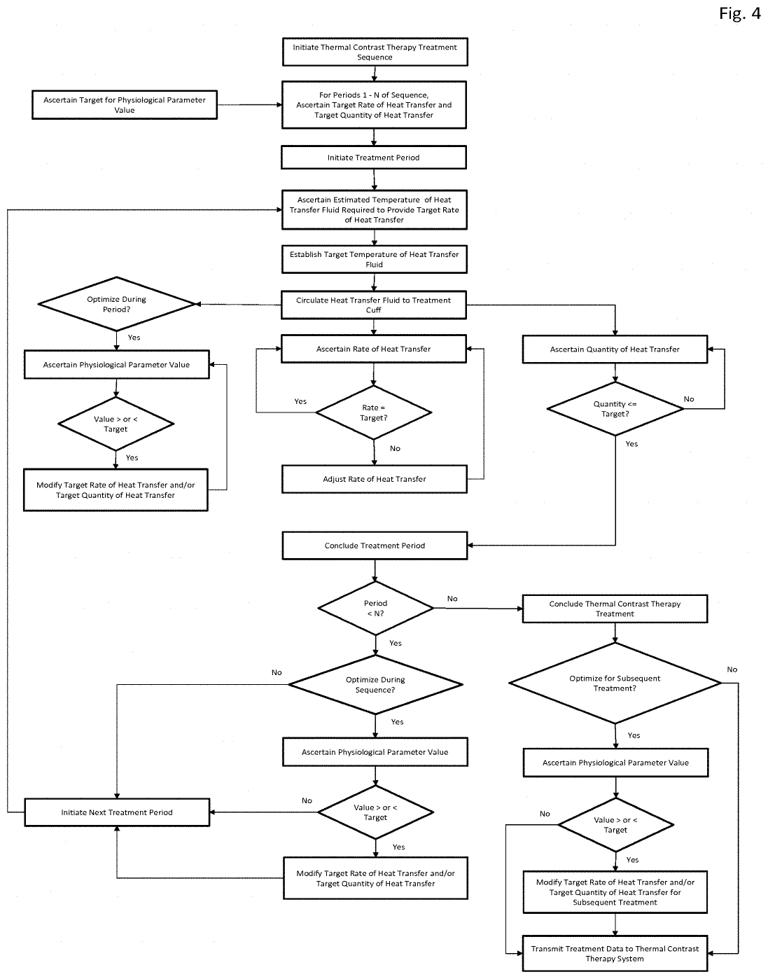

Referring to FIG. 4, a flow chart illustrative of an exemplary customized thermal contrast therapy treatment process, dynamically controlled to optimize a physiological parameter value is shown. The treatment may be configured to optimize a physiological parameter value during any one or more of the periods, and/or for subsequent periods within the sequence, and/or for subsequent treatments within a treatment program. During a treatment period, the thermal contrast therapy device ascertains whether the sequence calls for optimizing a physiological parameter value during the period. If so, the device ascertains the physiological parameter value, and if the value is less than or greater than a predefined target value, one or more measures of heat transfer (i.e., the rate of heat transfer and/or the quantity of heat transfer) to be provided during the period is modified in order to optimize the physiological parameter value. A predefined value corresponding to a physiological parameter may be one or more of: a target value, a minimum value, a maximum value, a range, an average, a standard deviation, an upper control limit, a lower control limit, a calculated value, a previous value, a change in the value, an increase from a previous value, a decrease from a previous value, a safety threshold, a deviation from any of the foregoing, an ascertained difference between a physiological parameter value and any of the foregoing and/or any other value desirable for dynamically controlling a thermal contrast therapy treatment sequence. A predefined value may be provide, for example, by a treatment provider or derived from a database. Alternatively or in addition, a thermal contrast therapy device and/or system may provide one or more preconfigured predefined values. Such preconfigured values maybe modified by a user and/or treatment provider.

When a treatment period has concluded, the thermal contrast therapy device ascertains whether the sequence calls for optimizing a physiological parameter value for subsequent periods within the sequence. If so, the device ascertains the physiological parameter value(s) exhibited during the concluded period and/or other previously concluded periods, and if the value(s) are less than or greater than a predefined target value, one or more measures of heat transfer to be provided during one or more subsequent periods in the sequence are modified in order to optimize the physiological parameter value.

When a treatment sequence has concluded, the thermal contrast therapy device ascertains whether the sequence calls for optimizing a physiological parameter value for subsequent treatment sequences within a treatment program. If so, the device ascertains the physiological parameter value(s) exhibited during the concluded sequence and/or other previously concluded sequences, and if the value(s) are less than or greater than a predefined target value, one or more measures of heat transfer to be provided during one or more subsequent treatment sequences are modified in order to optimize the physiological parameter value. At the conclusion of the treatment sequence, data associated with the sequence maybe transmitted to a thermal contrast therapy system for use in accordance with the present disclosure.

Dynamically controlled treatment sequences may include one or more automatic adjustments to the sequence. Automatic adjustments may occur at any desired time during the course of the sequence. A sequence may be automatically adjusted during the course of one or more cooling periods or heating periods within the sequence, for example, based on a physiological parameter value exhibited during the same period. Such automatic adjustment may be provided, for example, to attain a desired effect from a particular period within the sequence during which the one or more adjustments occur. Additionally, or in the alternative, one or more subsequent periods within a sequence may be automatically adjusted based on the effects of one or more prior periods, for example, a physiological parameter value exhibited during a previous cooling period or heating period. Automatic adjustments in subsequent period or periods may be provided, for example, to attain a desired effect from such subsequent period or periods. Automatic adjustments to a treatment sequence may be effected at any time during the course of treatment, for example, to attain a desired effect from the particular treatment. Automation adjustments may also be effected over the course of several treatments, for example, to attain a desired effect from a series of treatments.

In some instances, the particular settings which might be expected to produce a desired therapeutic effect may not be known, or may be uncertain. Given this, in some embodiments a thermal contrast therapy treatment sequences, and/or series of treatments within a treatment program may be configured to administer varying heating periods and/or cooling periods, treatment sequences, and/or overall treatment programs, while monitoring physiological parameter values, other therapeutic effects, and/or other factors related to the treatment, and then dynamically modify the treatment sequence(s) to optimize one or more physiological parameter values or other desired therapeutic effects. Such dynamic adjustments may be made to heating periods and/or cooling periods within a treatment sequence, to one or more treatment sequences making up a treatment program, and/or to an overall treatment program.

In some embodiments, a dynamically controlled treatment sequence is provided, wherein the dynamic control is configured to provide conditions derived from conditions of a prior treatment sequence. This may include reproducing or replicating conditions of a prior treatment, or adjusting one or more variables which may affect the treatment, a physiological parameter value, and/or a therapeutic effect of the treatment, for example, based at least in part on conditions of one or more prior treatment sequence.

In some embodiments, a thermal contrast therapy treatment sequence may be dynamically controlled based on a patient's comfort level. For example the sequence may be adjusted based on input as to whether the treatment feels painful or uncomfortable, too hot or too cold, or just right. Such patient input may be derived from one or more physiological parameter values, such as vital signs, or input may be provided directly by patient, for example via a user interface.

Those skilled in the art will appreciate that both feed-back and feed-forward control loops may be utilized in accordance with the dynamically controlled thermal contrast therapy treatment sequence of the present disclosure.

### Exemplary Customized Thermal Contrast Therapy Treatments

Customized thermal contrast therapy treatment sequences may be provided in accordance with the present disclosure based on any one or more variables, including the variables disclosed herein.

Customized treatment sequences may be provided based on the preference of the practitioner or therapist, or the patient receiving treatment. Treatment sequences may be developed on the basis of research and/or results attained by users associated with a thermal contrast therapy system.

Treatment sequences may be customized based on the area of the body being treated, the particular symptom or condition sought to be treated, or the particular desired therapeutic result. Treatment sequences may also be customized based on the particular body part(s) sought to be treated. As examples, customized treatment sequences may be provided for the hand, wrist, forearm, elbow, upper arm, shoulder, foot, ankle, calf, shin, knee, thigh, hips, pelvic region, abdomen, lumbar, mid-back, upper-back, neck, and cranium.

Treatment sequences may also be customized based on the particular conditions or symptoms sought to be treated. As examples, customized thermal contrast therapy may be provided to treat edema or swelling, fever, toxins, spasms, constipation, immune function inflammation, sprains, strains, general pain, neuropathy, arthritis, carpel tunnel, non-healing wounds, gangrene, migraine headaches, whiplash associated disorders, hair loss, muscular spasms, sinus pressure, disorders associated with lack of proper blood flow, bed sores, and respiratory ailments, among other things. Customized treatments may also be provided for patients with hypothermia or frostbite, for example, to enhance circulation in affected tissue.

As further examples, customized treatments may be provided to encourage healing of bones and tissue, and to rehabilitate injuries to bone, muscle, ligaments, tendons, and skin. Customized treatments may also be provided after an acute injury or surgery, for example, to reduce pain and swelling and promote healing. Customized treatments may be provided to athletes after or between training sessions to speed recovery or reduce delayed onset muscle soreness, by helping to flush lactic acid from sore muscles. Customized treatments may also be provided to relax joint tissue, such as ligaments and tendons, to increase range of motion. Customized treatments may also be provided for patients with spinal cord or nerve damage, for example, to enhance blood flow to the injured nerve tissue, or prevent or mitigate disputation of the tenuous blood supply.

Without wishing to be constrained by theory, it is believed that thermal contrast therapy performed on one area of the body may cause a sympathetic response in a different area of the body. For example, treatment performed on one limb (e.g., an arm or leg) may effect a therapeutic response in the opposite limb. Accordingly, in some embodiments customized treatments may be provided to effect a sympathetic response. The sympathetic response may include activation of the sympathetic nervous system, associated sensations associated with therapy in a limb opposite to the limb receiving direct treatment, increased blood flow, enhanced tissue healing and rehabilitation, and other desired therapeutic effects.

Customized treatments may also be an provided as a therapy for diabetes and related physiological complications, for example, gangrene, or for lymphedema or other disorders associated with vascular or lymphatic insufficiency, for example, Chronic Venous Insufficiency, venous stasis ulcers, post-mastectomy edema or chronic lymphedema, and for peripheral vascular disease or other circulatory deficiency syndrome, for example, arteriosclerosis, deep vein thrombosis, Buereger's disease, or thromboangiitis obliterans, or to positively influence the immune system.

Treatments may also be customized based on one or more desired therapeutic effects sought to be attained. As examples, therapeutic effects sought to be attained from thermal contrast therapy may include increased blood flow, increased blood oxygen levels, reduced inflammation, pain reduction, accelerated healing, increased medication effectiveness and focus, improved distal perfusion, improved arterial inflow, improved venous return flow, toxin removal, reduction of inflammatory kinins, and assistance to a chronically decompensated heart by complementing the heart cycle.

The total treatment time for a thermal contrast therapy sequence may range from a few minutes to several hours in duration, for example, from 10 minutes to 2.5 hours, or from 20 minutes to 75 minutes. As further examples, the total treatment time may be about 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, or any intermediate time period, or longer.

A treatment sequence may begin with either a heating period or a cooling period. Different therapeutic effects may be attained depending on whether therapy begins with a heating period or a cooling period. The duration of a heating period or a cooling period may range from a few seconds to several minutes. As examples, the duration of heating periods and/or cooling periods may range from about 5 seconds to 60 seconds, from about 1 minute to 5 minutes, from about 5 minutes to 20 minutes, or longer. A period may last, for example, about 5 seconds, 10 seconds, 15 seconds, 20 seconds, 25 seconds, 30 seconds, 35 seconds, 40 seconds, 45 seconds, 50 seconds, 55 seconds, 60 seconds, 65 seconds, 70 seconds, 75 seconds, 80 seconds, 85 seconds, 90 seconds, 95 seconds, 100 seconds, 105 seconds, 110 seconds, 115 seconds, 120 seconds, 125 seconds, 130 seconds, 135 seconds, 140 seconds, 145 seconds, 150 seconds, 155 seconds, 160 seconds, 165 seconds, 170 seconds, 175 seconds, 180 seconds, 185 seconds, 190 seconds, 195 seconds, 200 seconds, 205 seconds, 210 seconds, 215 seconds, 220 seconds, 225 seconds, 230 seconds, 235 seconds, 240 seconds, 5 minutes, 10 minutes, 15 minutes, 20 minutes, 25 minutes, 30 minutes, or any intermediate time period, or longer. Alternatively, in some embodiments, a cooling or heating period may be provided which comprises continuous application of hot or cold.

Transition times between a heating period and a cooling period, or vice versa, may range from a few seconds to several minutes, for example, from 5 seconds to 30 minutes. Transition times may be, for example, about 5 seconds, 10 seconds, 15 seconds, 20 seconds, 25 seconds, 30 seconds, 35 seconds, 40 seconds, 45 seconds, 50 seconds, 60 seconds, 1 minute, 2 minutes, 3 minutes, 4 minutes, 5 minutes, 10 minutes, 15 minutes, 20 minutes, 25 minutes, 30 minutes, or any intermediate time period, or longer.

Transition time may depend on the time required to flush heat transfer fluid from the applicable reservoir, through the tubing and into the cuff. Transition time may also depend on the desired therapeutic effect; for example, in some instances a rapid transition may be desired, whereas in other instances a more gradual transition may be desired. For example, when treating a physically fit patient for muscle spasms, a rapid transition may be provided, whereas an elderly person afflicted by poor circulation may be unable to withstand a high level of contrasting heat transfer, and as such, a mild transition may be required.

In some embodiments, a neutral period comprising a neutral temperature and/or rate of heat transfer may be provided between some or all of the heating periods and cooling periods. The neutral period may be provided as part of the transition between heating periods and cooling periods, or the neutral period may be treated as its own distinct period. The duration of a neutral period may range from a few seconds to several minutes. As examples, the duration of neutral periods may range from about 5 seconds to 60 seconds, from about 1 minute to 5 minutes, from about 5 minutes to 20 minutes, or longer. A neutral period may last, for example, about 5 seconds, 10 seconds, 15 seconds, 20 seconds, 25 seconds, 30 seconds, 35 seconds, 40 seconds, 45 seconds, 50 seconds, 55 seconds, 60 seconds, 65 seconds, 70 seconds, 75 seconds, 80 seconds, 85 seconds, 90 seconds, 95 seconds, 100 seconds, 105 seconds, 110 seconds, 115 seconds, 120 seconds, 125 seconds, 130 seconds, 135 seconds, 140 seconds, 145 seconds, 150 seconds, 155 seconds, 160 seconds, 165 seconds, 170 seconds, 175 seconds, 180 seconds, 185 seconds, 190 seconds, 195 seconds, 200 seconds, 205 seconds, 210 seconds, 215 seconds, 220 seconds, 225 seconds, 230 seconds, 235 seconds, 240 seconds, 5 minutes, 10 minutes, 15 minutes, 20 minutes, 25 minutes, 30 minutes, or any intermediate time period, or longer.

Heat transfer fluid temperatures during heating periods may range from about 85° F. to 130° F., or from about 100° F. to 110° F. The heat transfer fluid temperature during a heating period may be, for example, about 85° F., 90° F., 95° F., 100° F., 105° F., 110° F., 115° F., 120° F., 125° F., 130° F., or any intermediate temperature, cooler temperature, or warmer temperature. Other temperatures will also be apparent to the skilled artisan.

During cooling periods, heat transfer fluid temperatures may range from about 30° F. to 70° F., or from about 40° F. to 50° F. As further examples, cold heat transfer fluid temperatures may be about 30° F., 35° F., 40° F., 45° F., 50° F., 55° F., 60° F., 65° F., 70° F., or any intermediate temperature, cooler temperature, or warmer temperature. Other temperatures will also be apparent to the skilled artisan.

Transition periods may provide heat transfer fluid of any temperature which is between the temperature corresponding to the period preceding the transition period and the temperature which corresponds to the period following the transition period. For example, heat transfer fluid temperatures during a transition period may be about 30° F., 35° F., 40° F., 45° F., 50° F., 55° F., 60° F., 65° F., 70° F., 75° F., 80° F., 85° F., 90° F., 95° F., 100° F., 105° F., 110° F., 115° F., 120° F., 125° F., 130° F., or any intermediate temperature, cooler temperature, or warmer temperature. Other temperatures will also be apparent to the skilled artisan.

Transition periods may be configured to provide a customized transition curve. The transition curve between two periods may be rapid or gradual, may comprise various rates of change during the transition, and may include a neutral period comprising providing for a specified period of time, a desired rate of heat transfer, and/or a desired temperature for the heat transfer fluid, which is between that of the period preceding the neutral period and the period following the neutral period.

Flow rates of heat transfer fluids may range from about 10 mL/min to 1000 mL/min. For example, flow rates may be about 10 mL/min, 50 mL/min, 100 mL/min, 150 mL/min, 200 mL/min, 250 mL/min, 300 mL/min, 350 mL/min, 400 mL/min, 450 mL/min, 500 mL/min, 550 mL/min, 600 mL/min, 650 mL/min, 700 mL/min, 750 mL/min, 800 mL/min, 850 mL/min, 900 mL/min, 950 mL/min, 1000 mL/min, or any intermediate flow rate, higher flow rate, or lower flow rate. Flow rates may be increased where a high rate of heat transfer is desired, or decreased where a low rate of heat transfer is desired. In some embodiments, flow rate may be reduced to zero to provide a gradually declining rate of heat transfer. In some embodiments, flow rate may be increased to a point where the temperature difference between the fluid entering and exiting a treatment cuff approaches a target value, for example, a minimum value, or approximately zero.

Rates of heat transfer may range from about 0.5 BTU/min to 25 BTU/min. For example, the rate of heat transfer during a given period may be about 0.5 BTU/min, 1 BTU/min, 1.5 BTU/min, 2 BTU/min, 2.5 BTU/min, 3 BTU/min, 3.5 BTU/min, 4 BTU/min, 4.5 BTU/min, 5 BTU/min, 5.5 BTU/min, 6 BTU/min, 6.5 BTU/min, 7 BTU/min, 7.5 BTU/min, 8 BTU/min, 8.5 BTU/min, 9 BTU/min, 9.5 BTU/min, 10 BTU/min, 10.5 BTU/min, 11 BTU/min, 11.5 BTU/min, 12 BTU/min, 12.5 BTU/min, 13 BTU/min, 13.5 BTU/min, 14 BTU/min, 14.5 BTU/min, 15 BTU/min, 15.5 BTU/min, 16 BTU/min, 16.5 BTU/min, 17 BTU/min, 17.5 BTU/min, 18 BTU/min, 18.5 BTU/min, 19 BTU/min, 19.5 BTU/min, 20 BTU/min, 20.5 BTU/min, 21 BTU/min, 21.5 BTU/min, 22 BTU/min, 22.5 BTU/min, 23 BTU/min, 23.5 BTU/min, 24 BTU/min, 24.5 BTU/min, 25 BTU/min, or any intermediate rate, greater rate, or lesser rate. Other heat transfer rates will also be apparent to the skilled artisan.

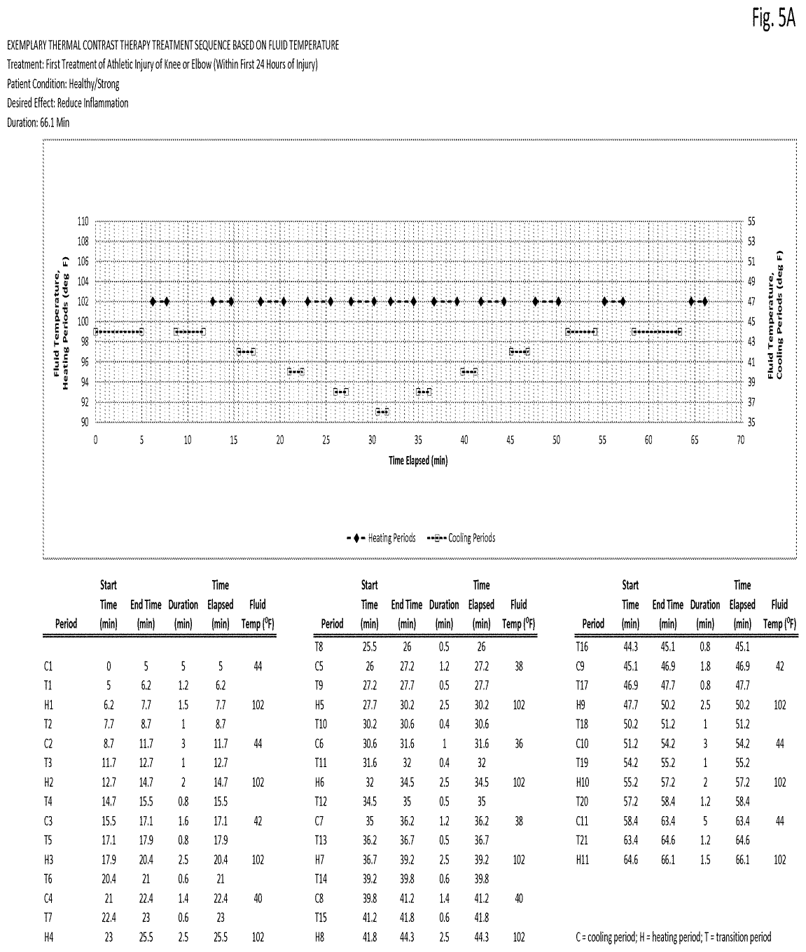

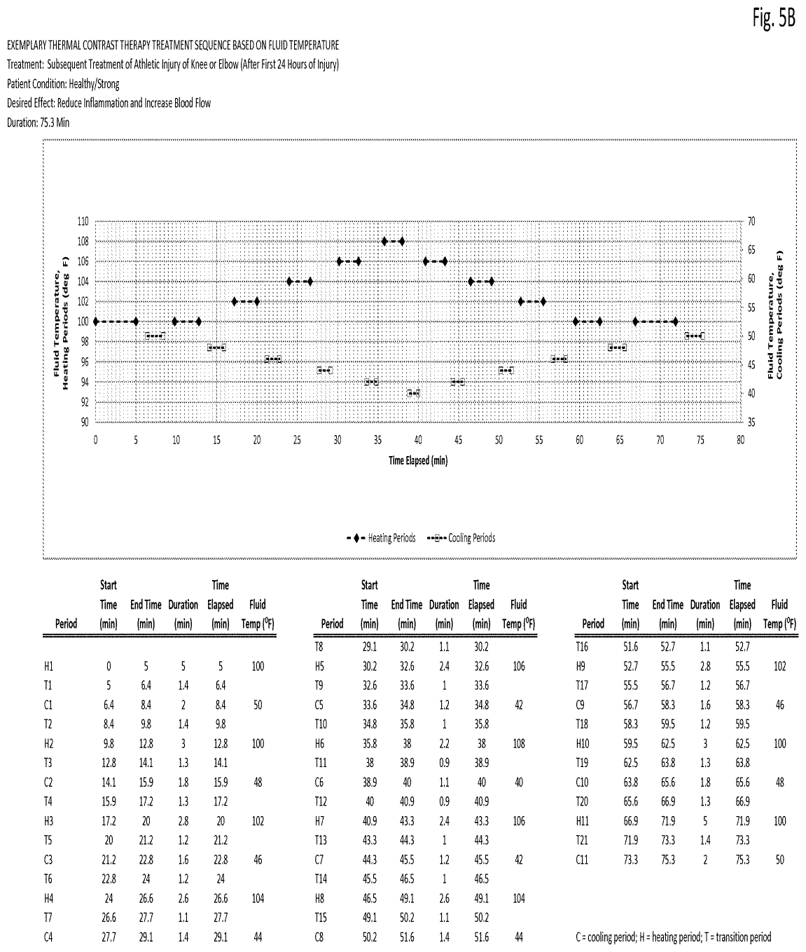

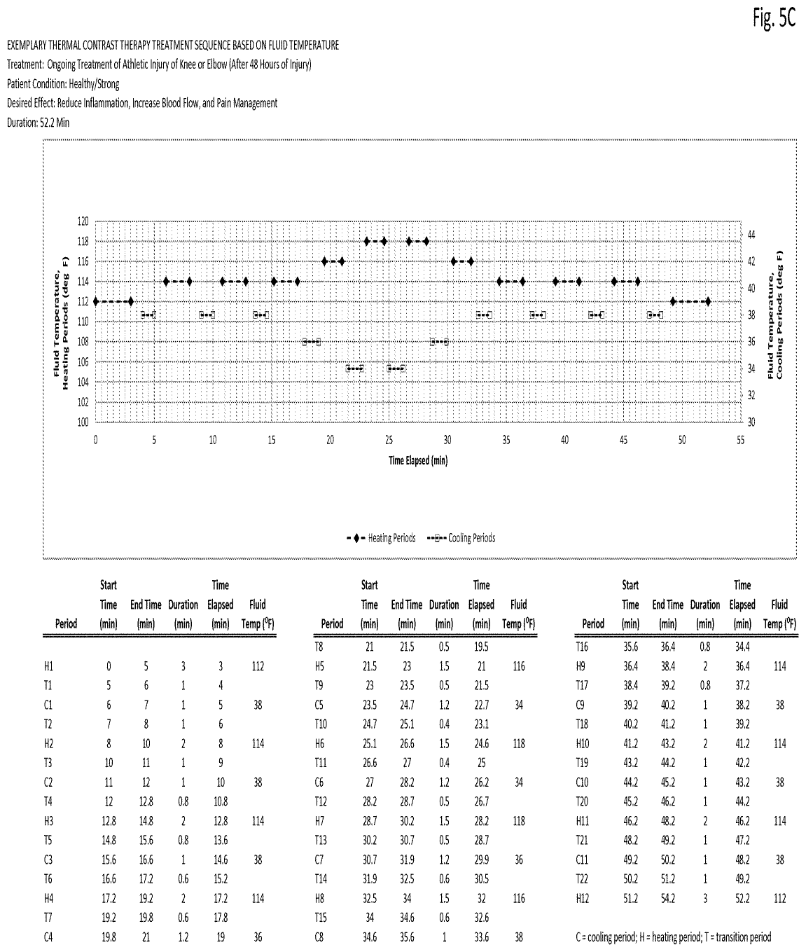

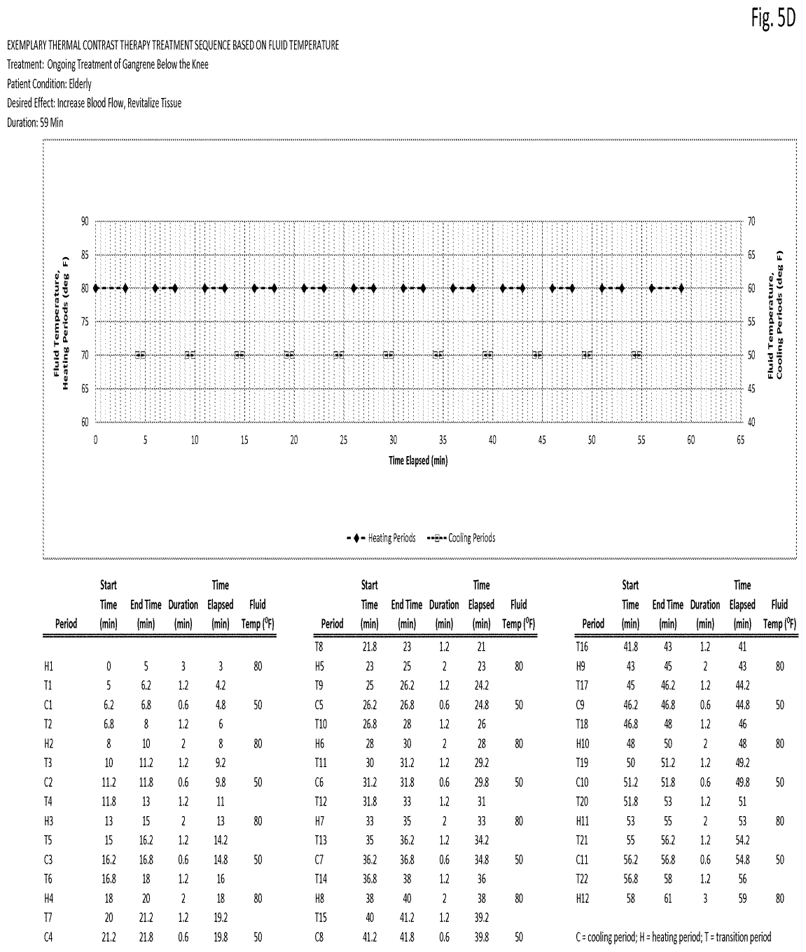

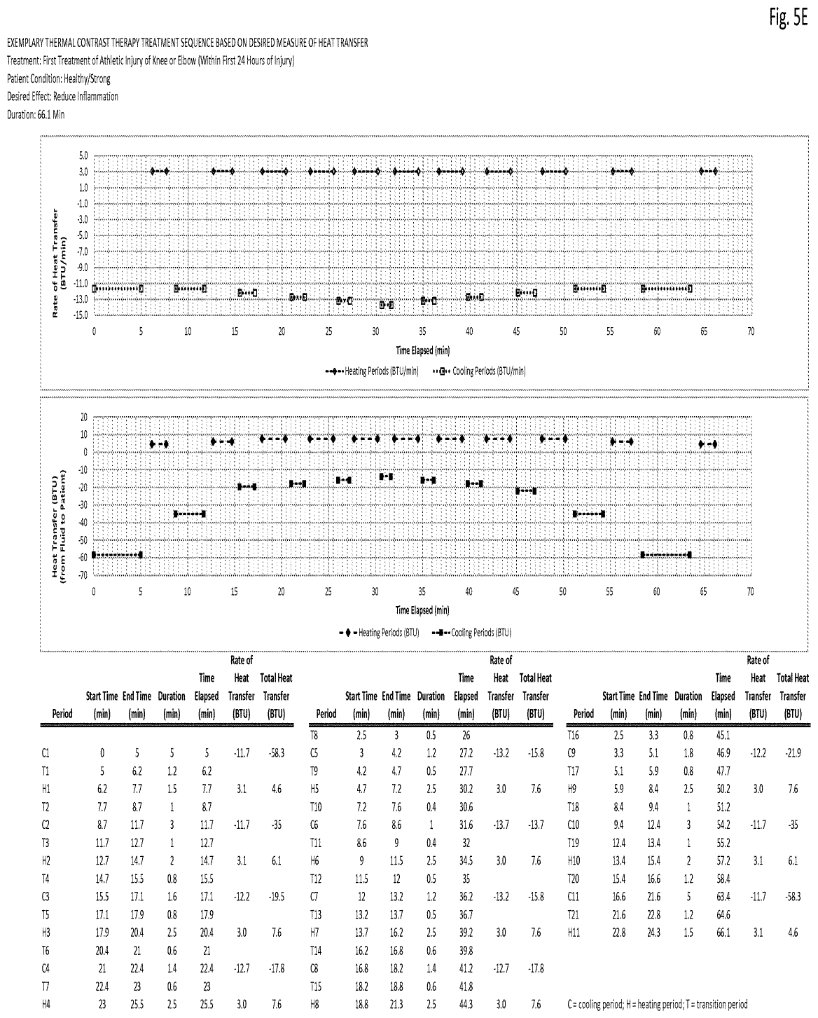

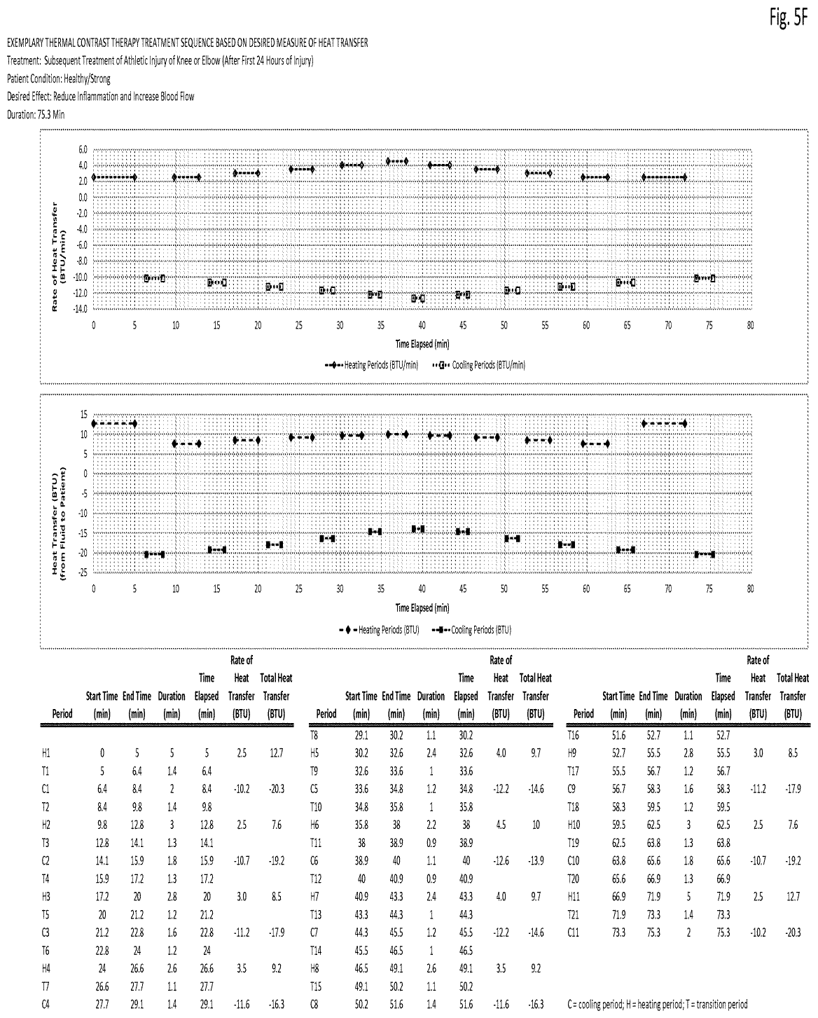

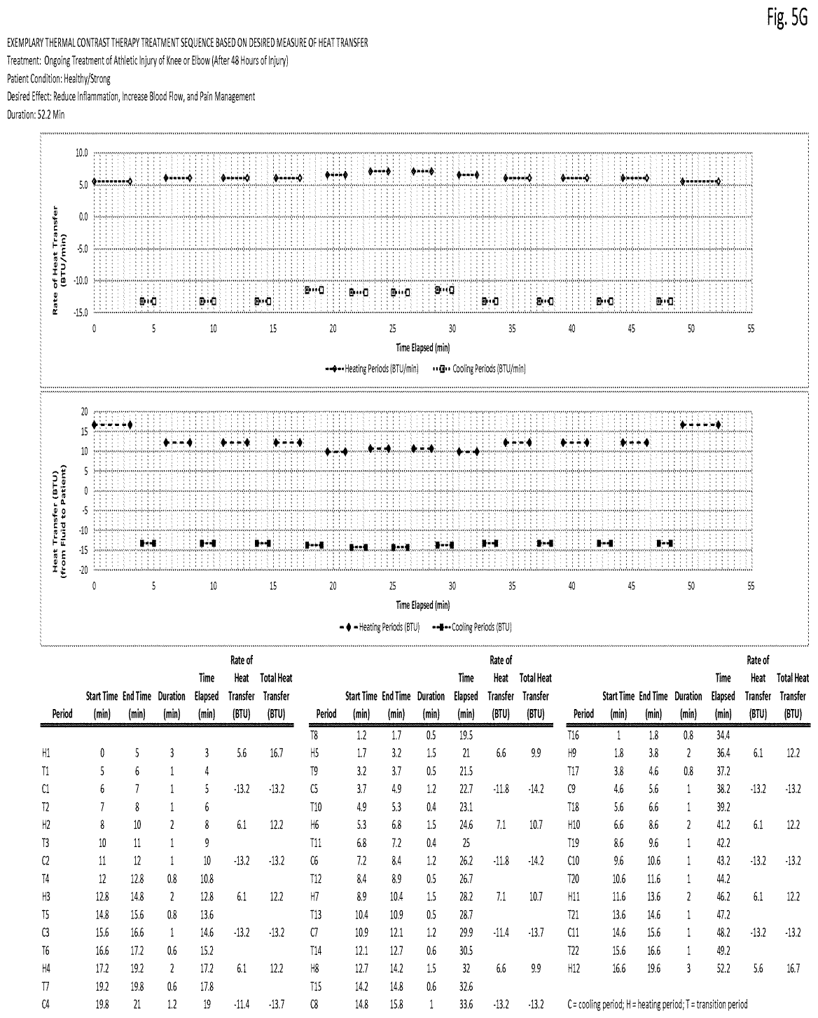

Referring to FIGS. 5A through 5G, a selection of exemplary thermal contrast therapy treatment sequences are shown. As depicted in FIGS. 5A, 5B, 5C, and 5D, a treatment sequence may be configured to provide a desired fluid temperature during the various treatment periods, and as depicted in FIGS. 5E, 5F, and 5G, a treatment sequence may be configured to provide a desired measure of heat transfer during the various periods. Additionally, treatment sequences may be configured to effect both a desired fluid temperature and a desired measure of heat transfer. Treatment sequences configured to effect a desired measure of heat transfer may provide for a desired rate of heat transfer and/or a desired quantity of heat transfer. Where a treatment sequence provides for a desired quantity of heat transfer, the duration of the treatment periods within the sequence may depend on the rate of heat transfer. In addition or as an alternative, treatment sequences configured to effect a desired rate of heat transfer may provide for treatment periods having a predefined time duration, where the desired quantity of heat transfer depends on such time duration.

FIG. 5A depicts an exemplary customized thermal contrast therapy treatment sequence configured to provide a desired fluid temperature during the various periods, for a first treatment of an athletic injury to a knee or elbow in a healthy/strong patient within the first 24 hours after the injury, to reduce inflammation. The treatment may be suitable, as an example, for an athlete to treat a knee injury that has occurred within 24 hours prior to treatment. The sequence depicted in FIG. 5A may be the first treatment of this injury, and as such the temperature contrast starts mildly and builds up through the treatment by decreasing the temperature of the heat transfer fluid cooling periods while maintaining constant temperature during the heating periods. Mid-way through the sequence, the region is allowed to calm with lesser contrast (by gradually increasing the temperature during the cooling periods), so as to reduce a sudden shock when the treatment it stopped.

FIG. 5B depicts an additional exemplary treatment sequence configured to provide a desired fluid temperature during the various periods. This treatment sequence may be suitable, as an example, for subsequent treatment of an athletic injury to a knee or elbow in a healthy/strong patient after the first 24 hours following the injury, to reduce inflammation and increase blood flow. For example, the sequence depicted in FIG. 5B may be provided as a subsequent treatment after previously having provided the sequence in FIG. 5A. Here, vasodilation and vasoconstriction may be enhanced by providing a gradually increasing fluid temperature during the heating periods and a gradually declining fluid temperature during cooling periods. Mid-way through the sequence, the region is allowed to calm with lesser contrast (by gradually increasing the temperature during the cooling periods and decreasing the temperature during heating periods), so as to reduce a sudden shock when the treatment it stopped.

FIG. 5C depicts yet an additional exemplary treatment sequence configured to provide a desired fluid temperature during the various periods which may be suitable for ongoing treatment during rehabilitation of an athletic injury to a knee or elbow. This treatment sequence may be suitable for a strong/healthy patient after the first 48 hours following the injury, to reduce inflammation, increase blood flow, and for pain management. For example, the sequence depicted in FIG. 5C may be provided as a subsequent treatment after previously having provided the sequence in FIG. 5B. Here, rapid consecutive alternating heating periods and cooling periods are provided, as indicated by the short transition times, and temperature differences between heating periods and cooling periods are large, with treatment temperatures intended to be at the threshold of a patient's tolerance so as to attain the maximum tolerable effect.

FIG. 5D depicts an exemplary thermal contrast therapy treatment sequence configured to provide a desired fluid temperature during the various periods, which may be suitable for treatment of gangrene below the knee. This treatment sequence may be suitable for an elderly patient in relatively poor physical condition, with a desired therapeutic effect of increasing blood flow and revitalizing tissue. Here, relaxed transitions and more modest temperature variations are used, given the patient's physical condition and sensitive nature of the tissue being treated.

FIG. 5E depicts an exemplary thermal contrast therapy treatment sequence configured to provide a desired measure of heat transfer during the various periods, which may be suitable for a first treatment of an athletic injury to a knee or elbow in a healthy/strong patient within the first 24 hours after the injury, to reduce inflammation. The treatment may be suitable, as an example, for an athlete with a knee injury that has occurred within 24 hours prior to treatment. The sequence depicted in FIG. 5A may be the first treatment of this injury, and as such the contrasting rates of heat transfer start mildly and builds up through the treatment by decreasing the temperature of the heat transfer fluid cooling periods while maintaining constant temperature during the heating periods. Mid-way through the sequence, the region is allowed to calm with lesser contrast (by gradually increasing the temperature during the cooling periods), so as to reduce a sudden shock when the treatment it stopped.

FIG. 5F depicts an exemplary thermal contrast therapy treatment sequence configured to provide a desired measure of heat transfer during the various periods, which may be suitable for subsequent treatment of an athletic injury to a knee or elbow in a healthy/strong patient after the first 24 hours following the injury, to reduce inflammation and increase blood flow. For example, the sequence depicted in FIG. 5F may be provided as a subsequent treatment after previously having provided the sequence in FIG. 5G. Here, vasodilation and vasoconstriction may be enhanced by providing a gradually increasing rate of heat transfer during the heating periods and a gradually increasing rate of heat transfer during cooling periods. Then, towards the end of the treatment sequence, the rate of heat transfer during cooling periods is gradually decreased to lessen vasoconstriction and to encourage increased blood flow in the tissue. Mid-way through the sequence, the region is allowed to calm with lesser contrast (by gradually decreasing the rate of heat transfer), so as to reduce a sudden shock when the treatment it stopped.

FIG. 5G depicts an exemplary thermal contrast therapy treatment sequence configured to provide a desired measure of heat transfer during the various periods, which may be suitable for ongoing treatment during rehabilitation of an athletic injury to a knee or elbow. This sequence may be suitable for a strong/healthy patient after the first 48 hours following the injury, to reduce inflammation increase blood flow, and for pain management. For example, the sequence depicted in FIG. 5G may be provided as a subsequent treatment after previously having provided the sequence in FIG. 5F. Here, rapid consecutive alteration between heating periods and cooling periods are provided, as indicated by the short transition times, and contrasting rates of heat transfer between heating periods and cooling periods are large, with such heat transfer rates intended to be at the threshold of a patient's tolerance so as to attain the maximum tolerable effect.

Each of the treatments sequences depicted in FIGS. 5A through 5G may be modified or combined with other sequences, features, or alternative embodiments in accordance with the present disclosure. For example, the sequences depicted in FIGS. 5A through 5D may be configured to provide a desired measure of heat transfer during the various periods rather than a desired fluid temperature. Additionally, any of the depicted sequences may be dynamically controlled in accordance with the present disclosure. For example, treatment sequence parameters may be automatically adjusted to optimize a physiological parameter value, and/or to effect a desired measure of heat transfer.

In the case of treating a recent injury, one or more of the heating periods may be replaced by a neutral period. Once the swelling has been controlled, heating periods may be introduced to increase contrast and therefore increase the blood flow to that region to provide pain reduction and to further encourage healing.

In the case of a weak or elderly patient, one or more of the cooling periods may be replaced by a neutral period. As the patient becomes increasingly accustomed to thermal contrast therapy, the cooling periods may be introduced.

Those skilled in the art will appreciate that various other treatment sequences are within the scope of the present disclosure, all as may be selected based on the preference of the practitioner or therapist or the patient receiving treatment, the area of the body sought to be treated, the particular symptom or condition sought to be treated, the particular desired therapeutic result, and/or other considerations.

### IV. User Profiles and Interfaces for Thermal Contrast Therapy Systems and Devices

### User Profiles

In some embodiments, it is desirable to provide user profiles for managing thermal contrast therapy treatments. User profiles may be used, for example, to store information about a patient and a patient's treatment history, to prescribe thermal contrast therapy treatment sequences for one or more users or treatment objectives, to define device and system settings corresponding to one or more users, and/or to define a selection of available features of a thermal contrast therapy system or device. Thermal contrast therapy treatment sequences, system settings, and available features may be defined by a user or characteristics of the user, or by a user's physician, therapist, or caretaker, etc. Treatment sequences, system settings, and available features of a thermal contrast therapy system or device may also depend on characteristics particular to a user, the selected or prescribed treatment, the particular area of the body sought to be treated, the particular symptom or condition sought to be treated, or the particular desired therapeutic result.

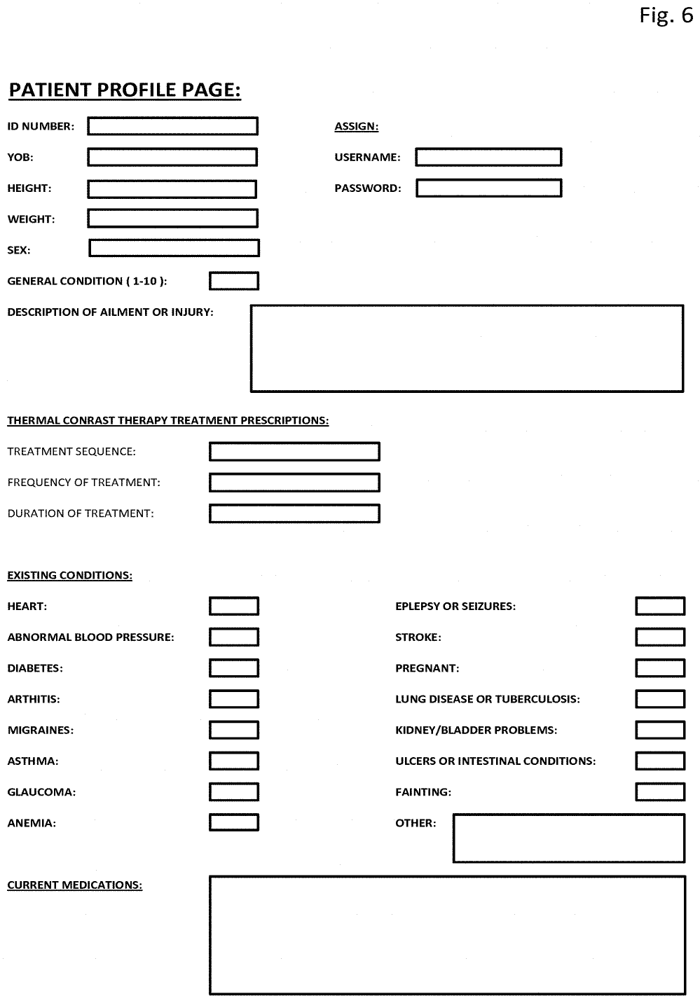

Referring to FIG. 6, an exemplary user or patient profile page for a thermal contrast therapy device or system is shown. As depicted in FIG. 6, a user profile may include information about the patient, such as personal information, medical history information, a description of the patient's ailment, injury, or condition sought to be treated, and information about the thermal contrast therapy treatment(s) which have been prescribed to the patient and/or selected by the patient, for example, via a thermal contrast therapy system.

In some embodiments, a selection of available features and operating conditions may be provided depending on information in a user's profile or other information. For example, a treatment provider, such as a physician or therapist may be provided a full selection of features, whereas a patient may be provided a more limited selection of features. Such user profiles may facilitate various thermal contrast therapy treatment sequences for use in clinical settings or for self-provided care at home or outside of a clinical setting. In some embodiments, a physician, therapist, or other caretaker may assign one or more prescribed thermal contrast therapy treatment sequences to a user profile, and the user desiring to self-provide therapy at home or outside of a clinical setting may select the one or more prescribed treatment sequences and cause the user's thermal contrast therapy device to perform the procedure. In some embodiments, the functionality of a user's thermal contrast therapy device may be limited such that the device will only perform one or more thermal contrast therapy treatment sequences prescribed to the user. The user's treatments and corresponding data, vital signs and other information may be recorded and stored in the user's profile, where it may subsequently be viewed by the user or the user's physician, therapist, or other care provider.

### User Interfaces

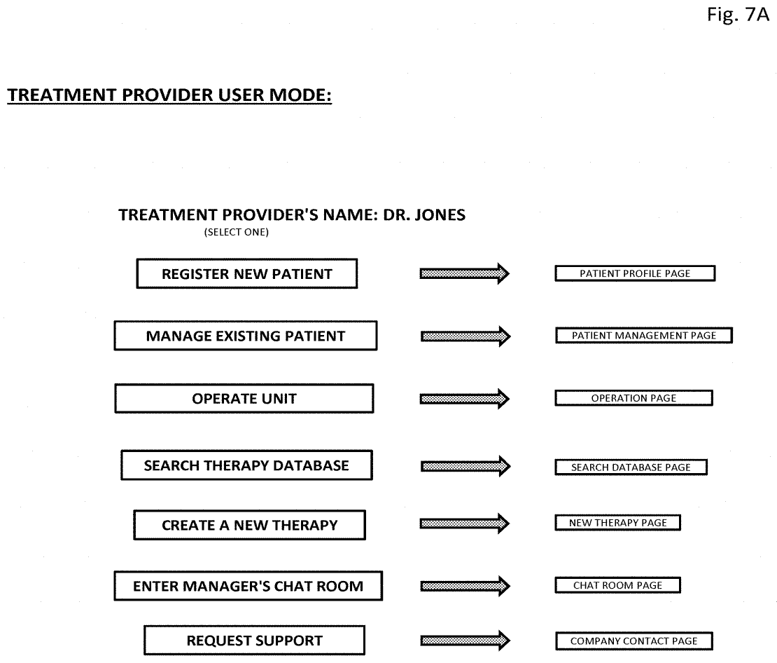

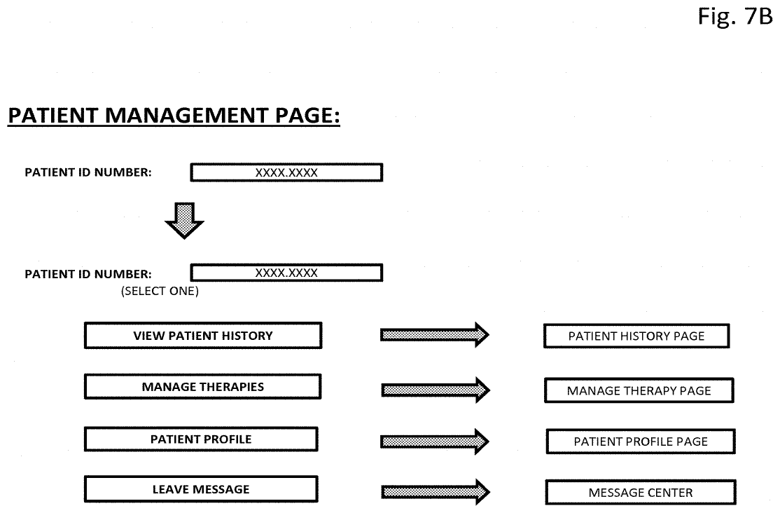

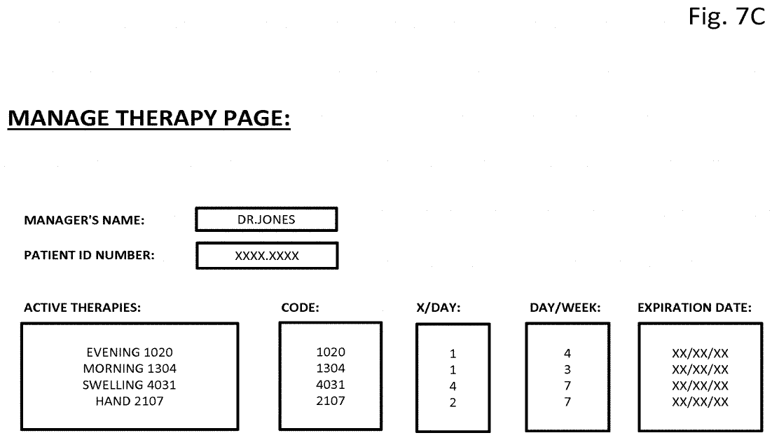

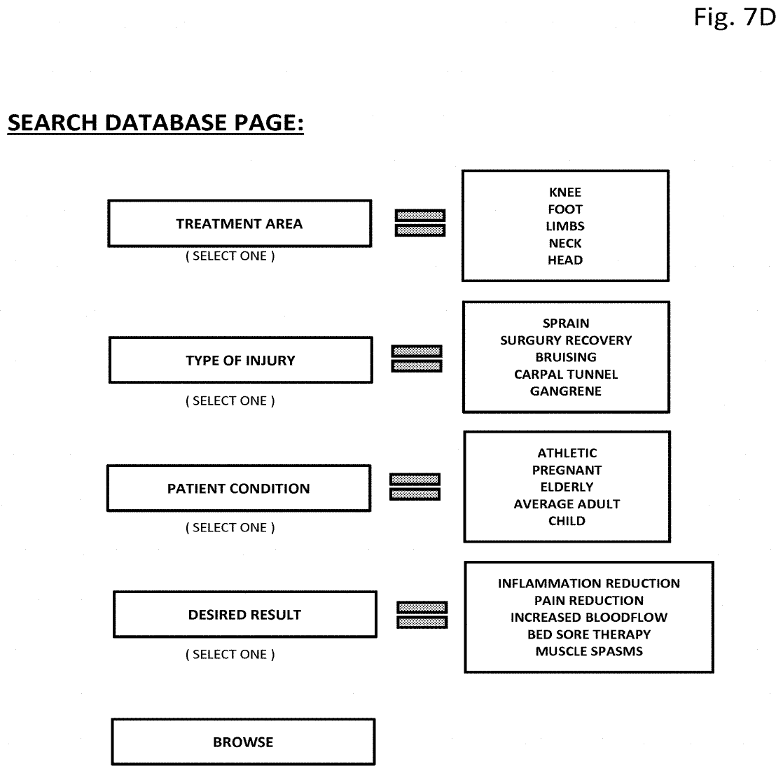

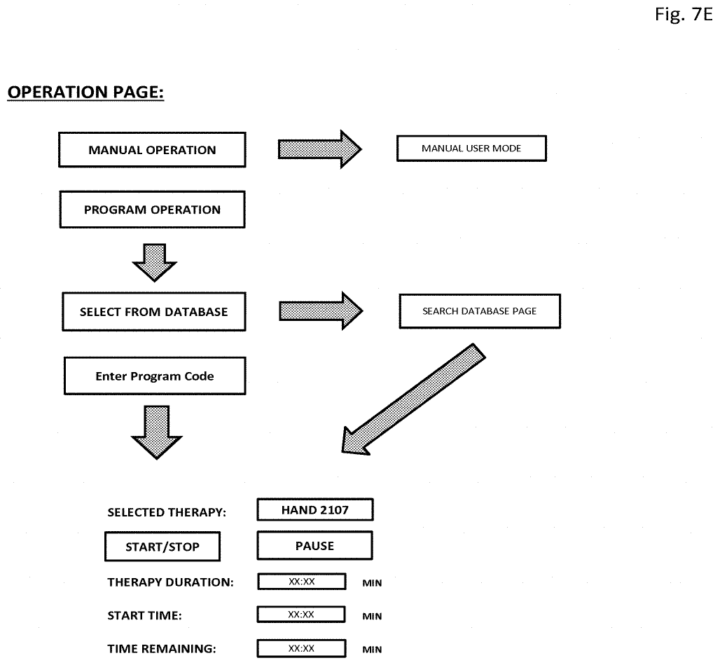

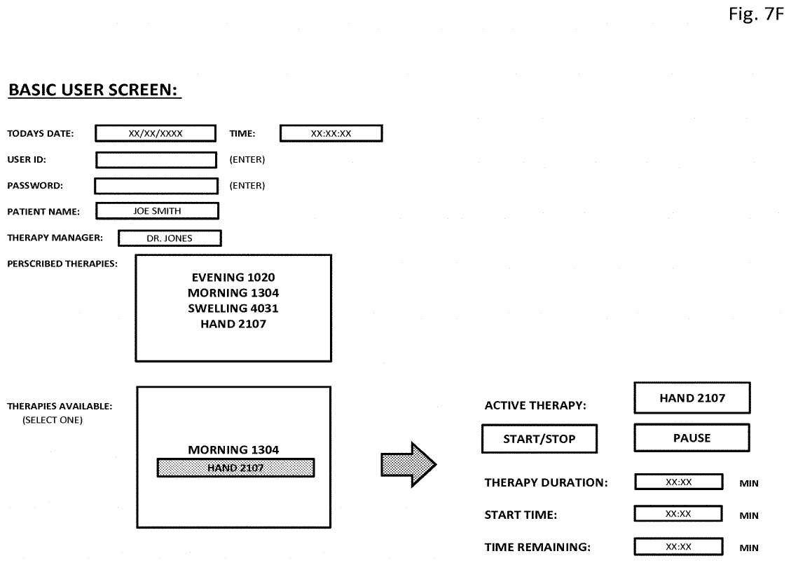

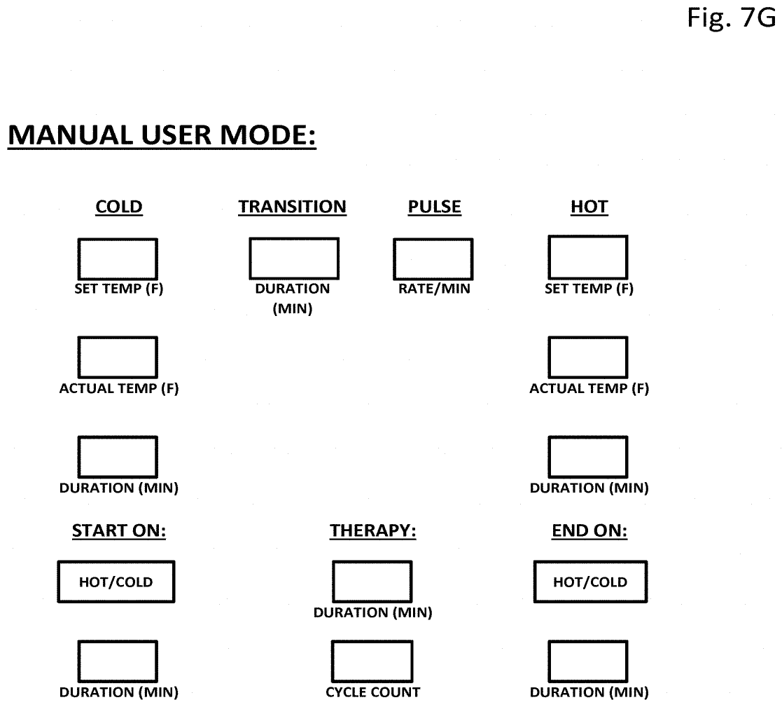

Various user interfaces may be provided to enable users to interface with thermal contrast therapy devices and systems in accordance with the present disclosure. Referring to FIGS. 7A through 7F, exemplary user interface pages for a thermal contrast therapy device and/or system are shown.

As depicted in FIG. 7A, an exemplary user interface may include a page configured to enable a treatment provider to utilize a thermal contrast therapy system. For example, a user interface page may be configured to enable a treatment provider to register new patients and manage care for existing patients. In some embodiments, a user interface may be configured to enable a treatment provider to remotely operate a thermal contrast therapy device. For example, a treatment provider may, via a user interface, initiate and monitor a thermal contrast therapy treatment sequence provided to a patient at a remote location. The user interface may also be configured to enable a treatment provider to communicate with the patient via voice, text, or video chat or the like, for example to confirm that the treatment is progressing satisfactorily and address questions or concerns that may arise. In some embodiments, a user interface may be configured to enable a treatment provider to search for a database to locate thermal contrast therapy treatment sequences which may be suitable for a patient, and/or to manually create a new treatment, which may be transmitted to a user's device and/or uploaded to the database for future use by the treatment provider or patient, and/or other treatment providers and patients. In some embodiments, a user interface may include a chat room for corresponding with other thermal contrast therapy treatment providers, subject matter experts, manufacturers, outside service providers, users, and the like.

As depicted in FIG. 7B, an exemplary user interface may include a page for a treatment provider to manage thermal contrast therapy treatments provided to patients receiving treatment via a thermal contrast therapy system. In some embodiments, a user interface may be configured to enable a treatment provider to view a patient's history and/or user profile, manage thermal contrast therapy treatments provided to the patient, and send a message to a patient.

As depicted in FIG. 7C, an exemplary user interface may include a page for a treatment provider to manage thermal contrast therapy treatments prescribed to a patient via a thermal contrast therapy system. For example, a user interface may be configured to enable a treatment provider to prescribe one or more treatment sequences and the time period and frequency with which they are to be administered.

FIG. 7D shows an exemplary user interface for searching a thermal contrast therapy system database for customized thermal contrast therapy treatments. As depicted in FIG. 7D, a user interface may be configured to enable searching based on a plurality of variables, including, for example, treatment area, type of injury or condition sought to be treated, the status or condition of a patient, and/or the desired therapeutic result sought to be attained. Treatments may be categorized in a database such as depicted in FIG. 7D based on any number of variables and each such variable may include any number of classifications. In some embodiments, a user interface may be configured to enable a patient to browse a complete listing or subset of treatments from a database.

FIGS. 7E, 7F, and 7G each show exemplary user interfaces for operation of thermal contrast therapy device. A user interface may be provided to enable operation of a thermal contrast therapy device to provide customized treatment sequences in accordance with the present disclosure. The user interface may be configured to enable operation of a thermal contrast therapy device from a remote location, for example via a thermal contrast therapy system, and/or locally, for example, via a touch screen or other interface integrated with the device, or via a handheld device configured to interact with the thermal contrast therapy device. In some embodiments, a user interface may be configured to allow a user to search a database and select a treatment program from the database to be performed by the user's thermal contrast therapy device. The treatment program selected by a user may be a program prescribed by a treatment provider, or any treatment program available in a database, for example, a treatment that matches a search query. As depicted in FIG. 7F, a user interface may be configured to display a user's prescribed thermal contrast therapy programs, and select a treatment sequence to be performed by the user's thermal contrast therapy device

As depicted in FIGS. 7E and 7F, an exemplary user interface may include a start/stop button to enable a user to start and stop a thermal contrast therapy treatment sequence, and/or a pause button, for example, to temporarily suspend the sequence. The user interface may be configured to display information about the treatment sequence, including information about the progress of the sequence, such as duration, start time, and time remaining. In some embodiments, a user interface may be configured to allow a user only to perform a prescribed sequence.

As depicted in FIG. 7G, a user interface may be configured to enable a user to manually operate a thermal contrast therapy device. Manual operation may be configured so as to enable a user to input settings for a thermal contrast therapy device and to cause the device to perform a treatment sequence based such settings. As depicted in FIG. 7G, manual input settings may include, for example, heat transfer fluid temperature settings, time durations for heating periods, cooling periods, and/or transition periods, frequency of fluid pulsation. Manual input settings may also include settings for desired rates of heat transfer, quantities of heat transfer, flow rates for heat transfer fluid, levels of compression. In some embodiments, manual operation may include manually starting and stopping a treatment sequence and/or treatment periods, or manually inputting a customized thermal contrast therapy treatment sequence. In some embodiments, manual input settings may be transmitted to a database associated with a thermal contrast therapy system for future use, for example, with subsequent treatment sequences.

### V. Other Embodiments

The foregoing detailed description of illustrative embodiments has set forth various embodiments of thermal contrast therapy systems, devices, treatment methods and/or processes via the use of block diagrams, flowcharts, and/or examples. Insofar as such block diagrams, flowcharts, and/or examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of ordinary skill in the art in light of the present disclosure.

In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control elements (e.g., feedback for sensing temperature; control heaters for adjusting temperature). A typical data processing system may be implemented utilizing any suitable commercially available components, such as those typically found in data computing/communication and/or network computing/communication systems.

The foregoing described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

All of the above U.S. patents, U.S. patent application publications, U.S. patent applications, foreign patents, foreign patent applications and non-patent publications referred to in this specification and/or listed in any Application Data Sheet, are incorporated herein by reference, in their entireties.

While various aspects and embodiments have been disclosed herein, other aspects and embodiments will be apparent to those skilled in the art. The various aspects and embodiments disclosed herein are for purposes of illustration and are not intended to be limiting. It is intended that the scope of this disclosure be defined by the following claims and their equivalents:

What is claimed is:

1. A method of providing customized thermal contrast therapy, the method comprising:
pumping fluid with a pump, to circulate the fluid through a treatment cuff comprising an entrance and an exit, the treatment cuff having been applied to a patient, in a sequence comprising a plurality of alternating cooling periods and heating periods, and a plurality of transition periods, occurring between the alternating cooling periods and heating periods, wherein at least one transition period is less than about 45 seconds;
ascertaining a measure of heat transfer between the fluid and the patient based at least in part on an input from a temperature sensor, the temperature sensor being configured to measure the temperature of fluid having circulated through the treatment cuff exit; and
changing the flow rate of the fluid circulating through the treatment cuff based at least in part on the measure of heat transfer.

2. The method of claim 1, wherein the specified measure of heat transfer comprises one or more of: a rate of heat transfer and a quantity of heat transfer, the rate of heat transfer being between about 0.5 BTU/min to about 25 BTU/min, and the quantity of heat transfer being between about 0.5 BTU to about 50 BTU.

3. The method of claim 1, the method further comprising receiving an input from an auxiliary device, the input indicative of a physiological parameter value exhibited by the patient, and changing the specified measure of heat transfer corresponding to one or more of the periods when the input corresponds to a predefined value, the physiological parameter value comprising one or more of: blood pressure, blood flow, and blood oxygen level.

4. The method of claim 3, the method further comprising changing the specified measure of heat transfer to optimize the physiological parameter.

5. The method of claim 3, the method further comprising changing the specified measure of heat transfer corresponding to the period during which the patient exhibits the physiological parameter value.

6. The method of claim 3, the method further comprising changing the specified measure of heat transfer corresponding to a period subsequent to the period during which the patient exhibits the physiological parameter value.

7. The method of claim 1, the method further comprising providing further customized thermal contrast therapy comprising subsequently providing one or more treatments having a prescribed sequence based, at least in part, on one or more physiological parameter values exhibited by the patient during then-previous treatments.

8. The method of claim 1, wherein each transition period comprises transitioning the rate of heat transfer effected between the fluid and the patient from about a first rate which corresponds to the period preceding the transition period to about a second rate which corresponds to the period following the transition period, and wherein at least one transition period is less than about 30 seconds.

9. The method of claim 1, wherein the method is effective to cause an increase in blood circulation in tissue proximate to the treatment cuff, and wherein changing the flow rate of the fluid is effective, at least in part, to optimize the magnitude of the increase.

10. The method of claim 1, wherein the method is effective to increase blood oxygen content in tissue proximate to the treatment cuff, and wherein changing the flow rate of the fluid is effective, at least in part, to optimize the magnitude of the increase.

11. The method of claim 1, further comprising providing a pulsation in the circulating fluid.

12. The method of claim 11, wherein the pulsation has a frequency and the method further comprises measuring the heart rate of the patient while providing pulsation and synchronizing the frequency with the heart rate of the patient.
